(19) United States
(12) Patent Application Publication
(10) Pub. No.: US 2025/0318789 A1
(43) Pub. Date: Oct. 16, 2025

(54) STROKE DETECTION BASED ON RESPONSE TO STIMULUS AND CONTINUOUS PHYSIOLOGICAL MONITORING

(71) Applicant: Kandu Health, Inc., Campbell, CA (US)

(72) Inventors: Lindsay Howard, San Jose, CA (US); Angela Alejandra Tejada Lazo, San Jose, CA (US); Kevin Thomas Looby, San Mateo, CA (US)

(21) Appl. No.: 19/173,543

(22) Filed: Apr. 8, 2025

Related U.S. Application Data
(60) Provisional application No. 63/632,875, filed on Apr. 11, 2024.
(60) Provisional application No. 63/672,510, filed on Jul. 17, 2024.
(60) Provisional application No. 63/705,927, filed on Oct. 10, 2024.

(51) Int. Cl. A61B 5/00; A61B 5/01; A61B 5/024; G16H 40/63

(52) U.S. Cl. CPC A61B 5/7282; A61B 5/01; A61B 5/02405; A61B 5/4884; A61B 5/6824; A61B 5/7267; A61B 5/7275; A61B 5/746; G16H 40/63; A61B 2560/0462; A61B 2562/0271

(57) ABSTRACT

A system for detecting an anomalous biologic event in a person includes a wearable device for monitoring skin surface sites of a person. The wearable device includes an electrode for contacting skin sites, an electronic stimulus source with a surface area to provide stimulus, and a sensor placed adjacent the skin sites to sense physiological data. A processor is coupled to the wearable device and is configured to: cause the stimulus source to generate a stimulus; excite the electrode to trigger monitoring the skin sites; cause operation of the sensor; receive bioelectrical data from each skin site; receive physiological data from the sensor; continuously compute a difference in the received bioelectrical data for a duration; compute a difference in the received physiological data at time intervals; and generate, based on the computation, an assessment including a likelihood of occurrence of the anomalous biologic event.

### FIELD

This disclosure relates generally to the field of disease detection and, more specifically, to stroke detection.

### BACKGROUND

A stroke results from the death of brain tissue due to disruptions of blood flow to the brain. An ischemic stroke happens when there is a blockage of blood flow to the brain, usually as the result of a blood clot. Hemorrhagic stroke happens when there is a rupture of a blood vessel in the brain, resulting in bleeding into the brain tissue and surrounding space.

There are many physiologic symptoms of stroke onset that vary depending on the location of the affected tissue. Early symptoms of an evolving stroke may be able to reduce or even resolve if the interruption of blood flow is resolved quickly before the tissue has died. One category of symptoms is disrupted vision, including blurred, dimming often likened to a curtain falling) or even complete loss of vision. Stroke patients (often also experience eye deviation or difficult with eye tracking.

Just as a stroke can affect the part of the brain that is associated with sight, it can also affect the parts of the brain that have to do with speech, comprehension and communication. Patients suffering from a stroke may exhibit slurred speech or garbled speech that renders them incomprehensible.

Another common symptom of stroke is weakness on one side of the body. This can manifest or partial or total paralysis of the side of the face, one arm, one leg, or the entire side of one's body.

Ischemic stroke is the most common type of stroke and is often painless when experienced, but hemorrhagic strokes are very painful, often being described as sudden onset of “the worst headache of one's life”. Often, many people's headaches are accompanied with a feeling of dizziness, nausea, and vomiting. Smell and taste can also be impacted during the onset of a stroke.

Anything that affects the brain, from trauma to stroke, has the potential for cognitive disablement. A feeling of confusion, or a constant second-guessing of ones' actions, can sometimes appear days before a stroke occurs.

Another common symptom of a stroke is the sudden onset of fatigue.

Stroke symptoms can vary in duration and occur with or without pain, which can make stroke detection difficult. Further, strokes can occur during sleep, making detection even more difficult. If a stroke does occur while the person is sleeping, it may not wake a person up right away. As a result, when patients wake up symptomatic, it is unclear whether the stroke just started or whether it has already been occurring during sleep.

If a stroke is detected and patients seek care quickly, there are many evidence-based interventions that can dramatically reduce the death and disability resultant from the disease. In severe ischemic strokes, every minute of delay to flow restoration is equated to the loss of a week of Disability Adjusted Life Years (DALYs). Despite these treatments being available, fewer than 20% of patients receive them. Even among patients that do receive intervention, outcomes are often suboptimal because of the delays to intervention. Stroke detection is difficult because stroke frequently doesn't hurt, mimics other health events, and is heterogeneous in its presentation. Improvements in detection of and care-seeking for stroke onset could dramatically reduce the death and disability associated with the disease.

Additionally, after a stroke is detected, a patient may need frequent monitoring over a prolonged period in order to identify and detect further signs of neurological decline so that appropriate treatment can be applied. Typically, this monitoring involves periodic manual assessment, such as with a neurocheck every few hours, over the course of approximately 72 hours after admission to a clinical environment. These assessments, however, are limited in that they can miss a significant amount of neurological decline due to their being periodic in nature and subject to a degree of subjectivity in interpretation due to their manual nature. Thus, improvements in detection of neurological decline after stroke onset could dramatically improve outcomes for neurological patients.

### SUMMARY

For purposes of summarizing the disclosure and the advantages achieved over the prior art, certain objects and advantages of the disclosure are described herein. Not all such objects or advantages may be achieved in any particular implementation. Thus, for example, those skilled in the art will recognize that the devices, systems, and methods may be embodied or carried out in a manner that achieves or optimizes one advantage or group of advantages as taught herein without necessarily achieving other objects or advantages as may be taught or suggested herein.

All of these implementations are intended to be within the scope of the devices, systems, and methods herein disclosed. These and other implementations will become readily apparent to those skilled in the art from the following detailed description of the implementations having reference to the attached figures, the devices, systems, and methods not being limited to any particular implementations disclosed.

In some implementations, a system for detecting an anomalous biologic event in a person can include: a wearable device including at least one physiological sensor configured to monitor at least one tissue site of a person and a stimulus source configured to apply a stimulus at the at least one tissue site to invoke a physiological response at the least one tissue site; and at least one hardware processor configured to: receive first data associated with the person; cause the stimulus source to generate a stimulus at the at least one tissue site; cause operation of the at least one physiological sensor to trigger monitoring of the at least one tissue site to generate second data, wherein the first data includes at least one type of physiological parameter data that is different from the second data; extract a set of features from the first data and the second data; generate a classifier based on at least two of the extracted set of features, wherein at least one of the extracted set of features comprises a metric corresponding to heart rate variability; and determine a likelihood of detection of an anomalous biologic event based at least in part on the generated classifier.

In some implementations, the stimulus source includes a heater and the second data includes skin temperature. In some implementations, the first data includes a data associated with heart activity of the person. In some implementations, the second data includes data associated with a skin temperature at two different locations on a body of the person.

In some implementations, a system for detecting an anomalous biologic event in a person can include: a wearable device including at least one physiological sensor configured to monitor at least one tissue site of a person and a stimulus source configured to apply a stimulus at the at least one tissue site to invoke a physiological response at the least one tissue site; and at least one hardware processor configured to: cause operation of the at least one physiological sensor to trigger monitoring of the at least one tissue site to generate first data; extract at least one feature from the first data; generate a classifier based on the extracted feature from the first data; compare the at least one feature to the generated classifier and a first threshold, wherein the first threshold is based at least in part on patient specific information; in response to a determination that the at least one feature satisfies the generated classifier and passes the first threshold, output an alert of a detection of the anomalous biologic event; and in response to a determination that the at least one feature does not satisfy the generated classifier and pass the first threshold, cause operation of the at least one physiological sensor to continue monitoring of the at least one tissue site to generate second data.

In some implementations, the stimulus source includes a heater and the second data includes skin temperature. In some implementations, the first data includes a data associated with heart activity of the person. In some implementations, the second data includes data associated with a skin temperature at two different locations on a body of the person. 9.

In some implementations, a system for detecting an anomalous biologic event in a person can include: a wearable device including at least one physiological sensor configured to monitor at least one tissue site of a person and a stimulus source configured to apply a stimulus at the at least one tissue site to invoke a physiological response at the least one tissue site; and at least one hardware processor configured to: receive first data associated with the person; cause the stimulus source to generate a stimulus at the at least one tissue site; cause operation of the at least one physiological sensor to trigger monitoring of the at least one tissue site to generate second data, wherein the first data includes at least one type of physiological parameter data that is different from the second data; extract a first and second set of features from the first data and the second data; generate a classifier based on at least the first and second set of features from the first data and the second data; determine a first likelihood of detection of the anomalous biologic event based on the first set of features and the generated classifier; determine a second likelihood of detection of the anomalous biologic event based on the second set of features and the generated classifier; determine an overall likelihood of detection of the anomalous biologic event based at least in part on the first likelihood and the second likelihood; and output an alert in response to the overall likelihood.

In some implementations, the at least one hardware processor is configured to cause the at least one physiological sensor to measure a distal temperature at a sampling rate of 16 Hz. In some implementations, the system includes generating an alert based at least in part on the likelihood of detection of an anomalous medical event. In some implementations, the tissue site includes a wrist. In some implementations, the stimulus source includes a heater and the second data includes skin temperature. In some implementations, the first data includes a data associated with heart activity of the person. In some implementations, the second data includes data associated with a skin temperature at two different locations on a body of the person.

In some implementations, a system for detecting an anomalous biologic event in a person can include: a wearable device including at least one physiological sensor configured to monitor at least one tissue site of a person; a memory; and at least one hardware processor configured to: cause to continuously measure, during a sleep period of the person by the at least one physiological sensor, a distal temperature at the at least one tissue site to generate a plurality of temperature data; extract a set of features from the plurality of temperature data; generate a classifier based on at least the set of features from the plurality of temperature data; and determine a likelihood of detection of the anomalous biologic event based at least in part on a the generated classifier.

In some implementations, an anomalous medical event is associated with increased variation. In some implementations, the at least one hardware processor is configured to cause the at least one physiological sensor to measure a distal temperature at a sampling rate of 16 Hz. In some implementations, the comparison includes: determining a presence or absence of an average increase in temperature values during the sleep period in the plurality of temperature data; determining a variation in temperature values during the sleep period in the plurality of temperature data; and generating the likelihood of detection of an anomalous medical event based at least in part on the absence of the average increase in temperature values and the variation in temperature values. In some implementations, an anomalous medical event is associated with an absence the average increase in temperature values during the sleep period.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various implementations, with reference made to the accompanying drawings.

These and other features, aspects, and advantages of the disclosure are described with reference to drawings of certain implementations, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 illustrates one implementation of a multivariate system for detecting an anomalous biologic event, such as a stroke.

FIGS. 2A-2C illustrate example implementations of a wearable system for detecting an anomalous biologic event.

FIG. 3 is a scatter plot showing an example feature of thermoregulation on the y-axis and a feature of heart rate variability on the x-axis.

FIG. 4 illustrates an example controller that may be part of a multivariate system for detection of an anomalous biologic event.

FIG. 5 illustrates example stroke detection processes that may be utilized by an example multivariate system described herein.

FIG. 6 illustrates a 3D plot showing an example comparison between triangular interpolation of N-N intervals in an ECG signal, a baseline skin temperature, and a baseline vasodilation.

FIG. 7 illustrates the 3D plot of FIG. 6 with outliers removed.

FIG. 8 illustrates a 3D plot showing an example comparison between fuzzy entropy, a baseline skin temperature, and a baseline vasodilation.

FIG. 9 illustrates the 3D plot of FIG. 8 with outliers removed.

FIG. 10 illustrates a 3D plot showing an example comparison between triangular interpolation of N-N intervals in an ECG signal, a baseline skin temperature, and a baseline vasodilation.

FIG. 11 illustrates the 3D plot of FIG. 10 with outliers removed.

FIG. 12 illustrates a 3D plot showing an example comparison between fuzzy entropy, a baseline skin temperature, and a baseline vasodilation.

FIGS. 13A-13N illustrate various boxplots of physiological parameters shown in the 3D plots of FIGS. 6 to 12.

The illustrated implementations are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various implementations. The inclusion of the following implementations is not intended to limit the disclosure to these implementations, but rather to enable any person skilled in the art to make and use the contemplated invention(s). Other implementations may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Although several implementations, examples, and illustrations are disclosed below, it will be understood by those of ordinary skill in the art that the devices, systems, and methods described herein extend beyond the specifically disclosed implementations, examples, and illustrations and includes other uses of the devices, systems, and methods and obvious modifications and equivalents thereof. Implementations are described with reference to the accompanying figures, wherein like numerals refer to like elements throughout. The terminology used in the description presented herein is not intended to be interpreted in any limited or restrictive manner simply because it is being used in conjunction with a detailed description of some specific implementations of the devices, systems, and methods. In addition, implementations can comprise several novel features. No single feature is solely responsible for its desirable attributes or is essential to practicing the devices, systems, and methods herein described.

The present disclosure may be understood by reference to the following detailed description. It is noted that, for purposes of illustrative clarity, certain elements in various drawings may not be drawn to scale, may be represented schematically or conceptually, or otherwise may not correspond exactly to certain physical configurations of implementations.

### Example Multivariate Stroke Detection System

Described herein are systems, devices, and methods for multivariate detection of stroke or at minimum, a deviation from baseline. Multivariate systems and methods may include using more than one, at least two, or a plurality of factors, markers, or other parameters to detect factors associated with an anomalous neurological event, such as a stroke, and/or neurological decline as a result of such an event. In some implementations, multivariate systems and methods can include using one parameter measured at multiple locations or positions or at multiple times (e.g., random or fixed intervals, on demand, automatically, continuously, etc.). In various implementations, multivariate systems and methods can include detecting a measured parameter symmetrically or asymmetrically. The measured parameter can include a functional parameter (e.g., gait, speech, facial changes, etc.); a biological parameter or marker (e.g., blood proteins, metabolites, etc.); a quantitative parameter (e.g., limb asymmetry, heart rate variability, etc.); a spatial (e.g., neck vs. chest; arm vs. leg; etc.) difference in one or multiple (e.g., 2, 3, 4, 5, 10, 15, 20, etc.) measured parameters; and/or a temporal difference in one or multiple measured parameters.

In some implementations, there can be an overlay of multivariate signals including two measurement data types, physiological (e.g., skin electric potential, Doppler flow signal anomaly, hyperhidrosis, cutaneous blood flow, brain perfusion, heartrate variability, etc.), and/or clinical manifestations or functional parameters (e.g., limb asymmetry, speech slur, facial droop, retinal abnormality, etc.). Clinical manifestations occur following stroke onset, but a faint signal from a clinical manifestation measurement combined with a physiological signal measurement can detect or predict stroke likelihood prior to stroke onset. Parameters that can be measured before, during, or after a stroke include quantitative parameters, functional parameters, and/or blood/fluid parameters. Any of the parameters shown/described herein can be measured asymmetrically, as described elsewhere herein. Exemplary, non-limiting examples of quantitative parameters include: volumetric impedance spectroscopy, EEG asymmetry, brain perfusion, skin/body temperature (e.g., cold paretic limb, up to 60 C colder or 16% colder than non-paretic limb), hyperhidrosis (e.g., greater than 40-60% increase on paretic limb), limb asymmetry, drift and pronation test, cutaneous blood flow, muscle tone, heartrate variability (e.g., decrease in spectral components by greater than 10×, lasting 3-7 days after stroke onset), facial surface electromyogram (EMG), cerebral blood flow (CBF), carotid artery stenosis, salivary cortisol, neuron specific enolase (NSE), salivary NSE, etc. Exemplary, non-limiting examples of functional parameters include: speech changes, speech comprehension, text comprehension, consciousness, coordination/directions, facial muscle weakness, arm weakness, body weakness (e.g., grip), leg weakness, foot weakness, unilateral weakness, difficulty walking, vertigo, sudden vision problems, limited visual field, altered gaze, thunderclap headache, nuchal rigidity (nape of neck), respiration, blood pressure (e.g., increase up to 60% in both systole (200 mHg) and diastole (140 mmHg)), etc. Exemplary, non-limiting examples of blood/fluid parameters include: CoaguCheck (Roche), HemoChron (ITC), iSTAT (Abbott), Cornell University, ReST (Valtari Bio Inc.), SMARTChip (sarissa Biomedical), etc.

In some implementations, multiple measurement locations (e.g., radial, brachial, etc. vessels) can be used to measure a difference in signal or data pattern among those locations compared to nominal, healthy location measurements or compared to an individual baseline as an input into a data processing module. For example, an individual baseline can be recorded over time and, when an adverse event occurs, a change (e.g., absolute or relative value) from baseline is determined unilaterally or bilaterally. In some implementations, after the adverse event occurs, a new baseline can be established. Further for example, blood pressure pulse varies depending on the location in the body, demonstrating that a slightly different signal is measured depending on location. For example, if only one location is measured, then changes over time are observed. If multiple locations are monitored and/or measured, then changes over time and changes relative to one another and/or a baseline can be used to identify a pattern or an asymmetric signal occurrence. In some implementations, an individualized baseline is further calculated based on a patient's health history (e.g., diabetes, heart-pacing, pre-existing stroke, etc.), demographics, lifestyle (e.g., smoker, active exerciser, drinks alcohol, etc.), etc.

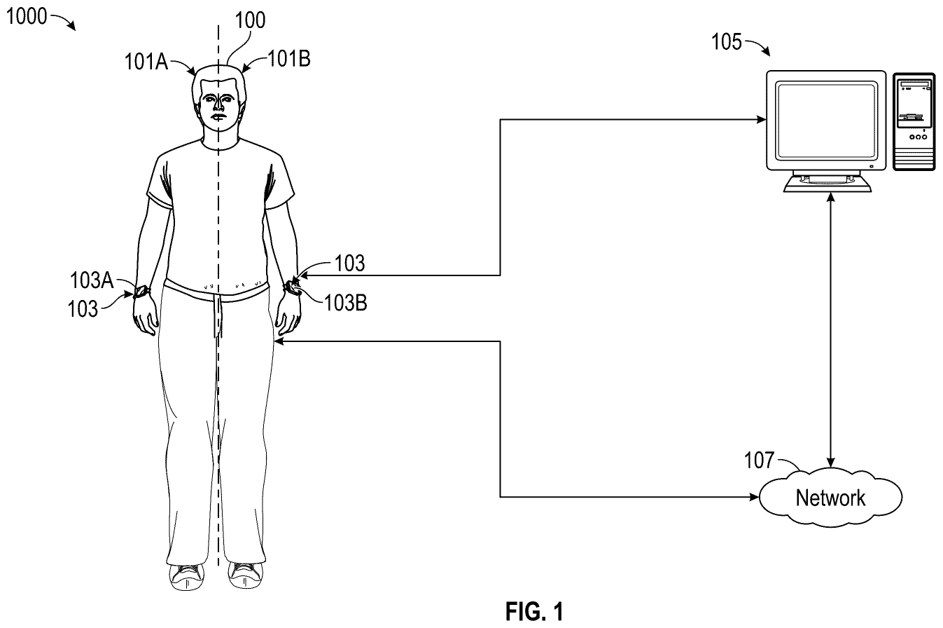

FIG. 1 illustrates an example multivariate stroke detection system 1000. As illustrated, the multivariate stroke detection system 1000 can include one or more physiological sensors 103 (e.g., a right physiological sensor 103A and/or a left physiological sensor 103B) configured to monitor a person 100 for an anomalous medical event, such as a stroke. The one or more physiological sensors 103 can include one or more wearable devices, such as a smart watch, smart ring, other wearable, and/or other device comprising one or more sensors configured to measure physiological data from a person 100. The one or more physiological sensors 103 can be configured to be in communication with one or more local computing devices 105 and/or one or more remote computing devices, such as a cloud or network 107. The system can include one or more hardware processors, such as can be part of one or more physiological sensors 103, one or more local computing devices 105, and/or network 107. The one or more hardware processors can be configured to calculate a risk associated with an anomalous medical event, such as of an anomalous medical event occurring presently, having previously occurred, or being likely to occur in the future, and generate an output based on the risk assessment. The one or more hardware processors can be configured to detect an anomalous medical event, such as a binary indication of a stroke having occurred or not having occurred.

The one or more physiological sensors 103 can be part of a single monitoring device configured to measure a systemic feature of a medical anomaly and/or the one or more physiological sensors 103 can be part of multiple devices, such as illustrated in FIG. 1, configured to measure a bilateral or otherwise non-systemic feature of a medical anomaly. A systemic feature can include, but is not necessarily limited to, a feature associated with a physiological condition as detected in a physiological signal or set of physiological signals indicative of a medical anomaly that can be detected in the physiological system of the person 100 as a whole, such as fever, increased blood pressure, changes in heart rate variability and others. A bilateral feature can include, but is not necessarily limited to, a feature of a physiological condition as detected in a physiological signal or set of physiological signals indicative of a medical anomaly that can be detected differently at different lateral sides, such as left lateral 101A and right lateral 101B of the body, such as asymmetry of vasodilation response, skin temperature, sweating, muscle activity, and others. Bilateral features typically relate to a degree of asymmetry of a physiological condition between the left lateral 101A and right lateral 101B of a person 100's body. The one or more physiological sensors 103 can be configured to measure systemic and/or bilateral features depending on their placement and use. In some examples, multiple of the one or more physiological sensors 103 can be configured to measure both systemic feature(s) and bilateral feature(s). In some examples, different of the one or more physiological sensors 103 can be configured to exclusively measure either a systemic or a bilateral feature. In some examples, one or more physiological sensors 103 can be configured to measure multiple different bilateral or systemic features. In some examples, one or more sensors 103 can be configured to measure individual or single bilateral or systemic features.

The one or more physiological sensors 103 can be configured to passively and/or actively measure features. For example, in some examples, the one or more physiological sensors 103 can be configured to measure the physiological condition of the person 100 without or without significantly influencing the physiological response or condition of the person. In addition or in the alternative, the one or more physiological sensors 103 generate a stimulus to provoke a physiological response or condition in the person 100, such as vasodilation or skin temperature change. The one or more physiological sensors 103 can be configured to then detect the response after application of the stimulus. In some examples, a different device can be configured to apply the stimulus than to detect the physiological response.

The one or more physiological sensors 103 can be configured to measure features based on user input and/or based on a continuous, periodic, or episodic schedule. In some examples, the one or more physiological sensors 103 can be configured to initiate detection of a measurement based on a user input, such as an indication of a desire to begin a test for a medical anomaly or based on a schedule set by a user. In some examples, the one or more physiological sensors 103 can be configured to perform a measurement based on continuous monitoring, semi-continuous monitoring, periodic or episodic monitoring. In some examples, the schedule can be different based on detected patient activity or movement. For example, the schedule can include less frequent sampling during periods of active movement where sensor signals can be less reliable. In some examples, a “continuous” monitoring can be every 10 minutes, every 5 minutes, every 1 minute, less than 1 minute, or a frequency shorter or greater than those frequencies. The periodicity and/or schedule of monitoring can be predetermined, based on user input, and/or determined based on parameters associated with the person 100. For example, if a system determines that the person 100 has an elevated risk factor of stroke, the system can increase the periodicity of monitoring or switch to continuous monitoring. In another example, if a clinician identifies a level of risk of a medical anomaly, the clinician can set a monitoring schedule in association with that risk. Advantageously, the continuous monitoring and/or customizability of a monitoring system and/or schedule can help reduce strain on clinician resources, such as in a hospital environment, and facilitate closer monitoring of a person 100 than might otherwise be available as a result of manual monitoring.

In some examples, one or more features and/or predetermined risks can be tracked and/or input into the system separately from the one or more physiological sensors 103. For example, one or more features related to blood work, visual inspection of a person 100, medical record information, historical physiological data, the like or a combination thereof can be accessed and/or utilized by the system to generate a risk assessment and/or detect a medical anomaly, such as a stroke. Data from one or more sources, such as historical and present data from a single source or type of source, historical and/or present data from varied sources, and/or user inputs can be utilized in combination and/or separately to generate an assessment of a person 100. In some examples, such data can be weighted and combined to generate a single assessment. In some examples, data can be separately evaluated based on a parameter, such as source type or data type (e.g., past clinician assessments can be grouped or historical physiological data can be grouped with present physiological data) to generate multiple assessments.

The system 1000 can include one or more hardware processors configured to calculate a likelihood or risk associated with an anomalous medical event, such as of an anomalous medical event occurring presently, having previously occurred, or being likely to occur in the future, and generate an output based on the risk assessment. The one or more hardware processors can include one or more hardware processors associated with local or network devices associated the system 1000, such as the one or more local computing devices 105, network devices 107, and/or one or more physiological sensors 103. The one or more hardware processors can be part of the same device or separate devices and can be local and/or in the cloud.

The one or more physiological sensors 103, one or more local computing devices 105, and/or network 107 can be configured to communicate data related to the person 100 and/or multivariate stroke detection system 1000, including but not limited to physiological information collected by, for example, the one or more physiological sensors 103, medical records information, clinician and/or other user inputs related to the person 100 and/or multivariate stroke detection system 1000, alerts and/or other outputs related to the person 100.

In some examples, the one or more hardware processors can be configured to process raw signal from the one or more physiological sensors 103 and/or other devices for feature analysis. For example, the one or more hardware processors can be configured to window a signal, perform signal correction, remove noise, validate a signal, interpolate missing or inaccurate data, smooth signals, the like or a combination thereof. In some examples, noise removal can include monitoring and/or tracking one or more additional signals and/or features of additional signals, such as skin temperature signals, motion signals, or other features and/or signals by which a noise reference can be determined for noise removal and/or signal correction. Additionally or alternatively, the one or more hardware processors can be configured to detect one or more features in signals received from the one or more physiological sensors 103 or other inputs. For example, the one or more hardware processors can be configured to determine one or more features based on the physiological signals and/or other data.

The one or more hardware processors can be configured to determine an output based on determined features in the physiological signals and/or other data. The one or more hardware processors can be configured to extract one or more features utilizing any number of classification methods, including but not limited to model fitting or other fixed classification method and/or machine learning or other learning and/or personalizable or customizable to the patient classification method, or some combination thereof. Features used to generate an output, such as a likelihood or risk associated with an anomalous medical event, can be one or more features or pairs of features. Features can be utilized or selected based on correlation of features with likelihood or risk associated with an anomalous medical event, such as heart rate variability, skin temperature, blood pressure, and/or vasodilation. Features can be utilized or selected based on other factors, such as patient medical history or other information that can impact the correlation between the feature and anomalous medical event or for another reason, such as hardware considerations, sensor signal quality, or the like.

An output can include, but is not limited to one or more risk assessment(s), physiological data, alert(s), report(s), treatment recommendation(s), control signal(s) to monitoring and/or treatment device(s), and/or a combination thereof. The output can be output to patient monitoring and/or clinician devices, personal computing devices, the cloud, an electronic medical record, emergency contacts, emergency services, and/or other devices and/or users. Outputs, such as alerts, can be communicated so as to call attention to the person 100 such that the person 100 can receive appropriate medical attention. For example, an alert can be audible to facilitate persons in the vicinity of the monitored person 100 to understand that help can be needed. In another example, an alert can be pushed to a clinician device or patient monitor to facilitate notifying a treating physician that they should initiate treatment. In some examples, an alert can be output to emergency services in order to facilitate faster treatment of the person in a high risk situation.

An output can be different based on a level of risk and/or other calculation performed by the one or more hardware processors. For example, if a high likelihood of stroke is detected, the system 1000 can be configured to contact an emergency service provider. If a moderate likelihood of stroke is detected, the system 1000 can be configured to instruct a person to seek help, such as an emergency department or other urgent care provider. If a low to moderate likelihood of stroke is detected, the system 1000 can be configured to instruct a person to provide additional input for further analysis and/or evaluation by the system 1000, such as answering of questions, performing tests, the like or a combination thereof. In some examples, the system 1000 can additionally and/or alternatively contact an emergency contact and/or healthcare provider or emergency services provider if a likelihood of stroke is above a certain threshold. The system can determine a level of likelihood of stroke or other anomalous medical event by, for example, comparing a numerical identification of risk (such as can be based on feature identification discussed herein) to a threshold or a plurality of thresholds.

The multivariate system 1000 described herein can be applicable to a variety of patient monitoring environments, such as environments where quick, short term assessments are utilized and environments needing longer term monitoring. For example, emergency medical services can utilize a spot check assessment using systems and methods described herein to determine a risk of a medical anomaly occurring or having occurred or otherwise identify the occurrence of a medical anomaly. In another example, at home or longer term hospital monitoring can utilize continuous and/or episodic or periodic monitoring to determine a risk of a medical anomaly occurring or identify the occurrence of a medical anomaly when a person is already identified as at risk.

### Example Sensing Device

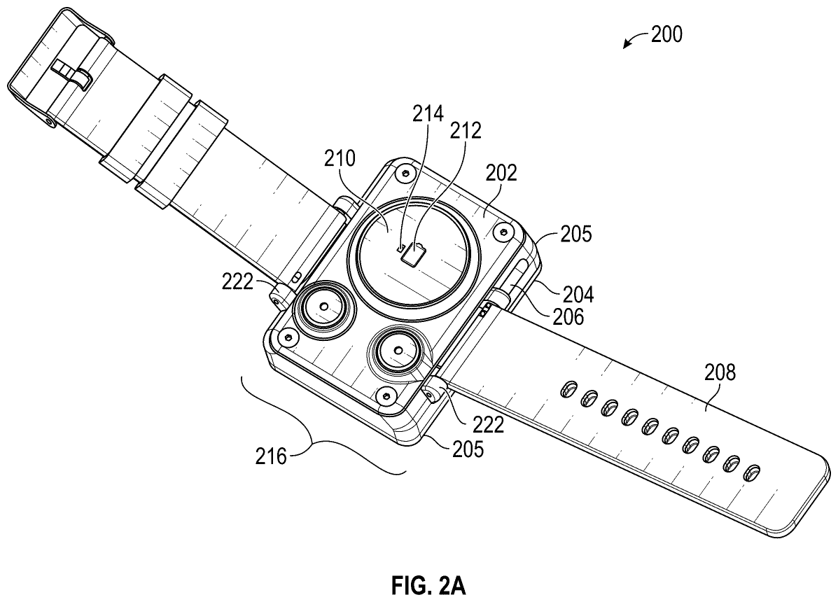

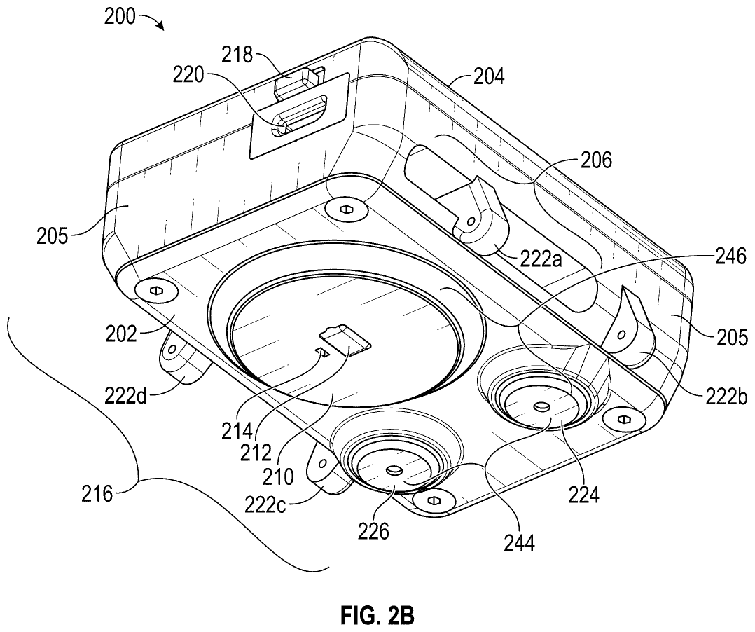

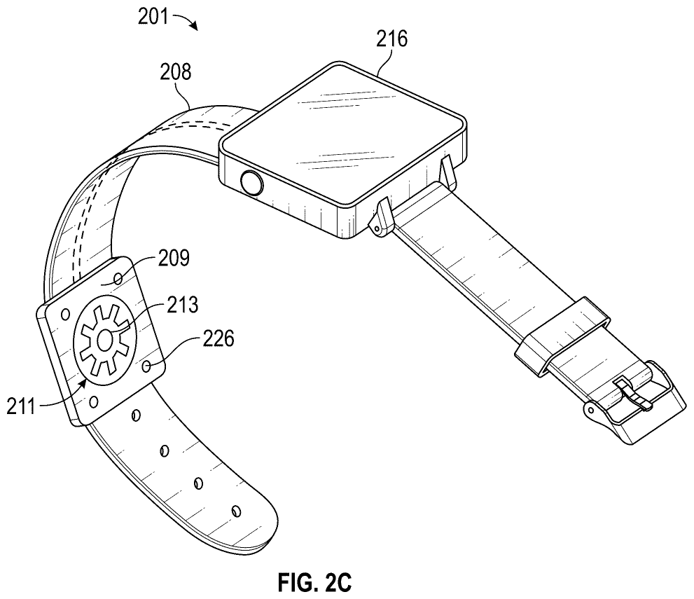

FIGS. 2A-2C illustrate an example of a physiological sensor 103, such as can be part of a multivariate stroke detection system 1000 described with reference to FIG. 1. In the illustrated example, the one or more physiological sensors 103 can be configured to enable measurement of a feature, such as skin temperature or vasodilation. However, other types of sensors and/or features are also possible. Further details and/or examples of one or more physiological sensors that can be utilized as part of a multivariate stroke detection system 1000 are disclosed in Intl. Pub. No. PCT/US2022/071701, filed Apr. 13, 2022, entitled SYSTEMS AND METHODS FOR MULTIVARIATE STROKE DETECTION, the entirety of which is hereby incorporated by reference herein in its entirety.

As discussed herein, applying heat stress to a portion of the skin can enable detection of vasodilation response. Accordingly, systems and methods described below enable detection of impaired vasodilation in a form factor that improves continuous anomalous cardiac event monitoring. In some implementations, as shown in FIGS. 2A-2C, a system or device 200, 201 for detecting an anomalous biologic event can function to heat a skin surface and measure a vasodilation response of the skin surface. The system or device 200, 201 can further function to measure one or more additional parameters, biologic signals, etc. In some implementations, the system or device 200, 201 can use a measured bioimpedance (BioZ) to validate or invalidate a vasodilation measurement from another sensor on system or device 200, 201.

In one example, a system or device 200, 201 for detecting an anomalous biologic event can include a body 216 having a first surface 204 opposite a second surface 202 in contact with a skin surface of a person. The first surface 204 and second surface 202 can be coupled via one or more or a plurality of sidewalls 205. For example, one or more sidewalls 205 can extend from a perimeter of the first surface 204 and couple to a perimeter of the second surface 202. The first surface 204 and/or second surface 202 can include one or more sensors positioned thereon. For example, one or more sensors on the first surface 204 can measure an environment of the user wearing or using the wearable system, and one or more sensors on the second surface 202 can measure one or more properties, features, or characteristics of the skin surface of the user and thus the user itself. Alternatively, the first surface 204 can include one or more sensors or imagers or cameras for assessing a facial region of a user, for example, via a FAST test (e.g., using facial recognition, movement analysis, and/or speech recognition to assess a person's facial symmetry, arm movements, and/or speech patterns).

A wearable device 200 can be secured to a user, for example a limb of a user or a skin surface of a user, via a band 208, for example a tensionable band, which will be described in greater detail elsewhere herein. The band 208 can be adjustable such that the wearable device 200 can be cinched or tensioned to promote greater contact and thus coupling between the wearable device 200 and the skin surface or tension released to reduce contact or coupling between the wearable device 200 and the skin surface. As shown in FIGS. 2A-2B, a band 208 can be coupled to a body 216 of a wearable device via one or more connectors 222 (e.g., connects 222a-222d). For example, a band 208 can couple to a body 216 of a wearable device via a connector 222 that includes one or more pin joints, a snap fit connection to the band 208, a slide and fit connection to the band 208, etc. When the tensionable band 208 is coupled to the body 216 via connectors 222, the tensionable band can be centered with respect to one or more sensors positioned on the second surface, so that there is sufficient coupling between the sensors and the skin surface.

A wearable device 200 can include a heat source 210 in communication with the skin surface. The heat source 210 can be configured to heat the skin surface to a target temperature or a pre-determined temperature. The heat source 210 can be a heating element; thin film resistance flexible heater (e.g., a single printed layer of resistive ink on polyimide film); polyimide heater; optical heater (e.g., a laser), etc. In other implementations, the heat source 210 is an environmental heat source, for example a warm room, warm environment (e.g., under the covers, hot day, etc.). In such implementations, the stimulus can be a change in environmental temperature, for example, from a warm environment to a cool environment or a cool environment to a warm environment. In some implementations, a heat source 210 is positioned on a second surface 202 of the body 216, so that there is coupling or contact between the heat source 210 and a skin surface. Alternatively or additionally, a heat source or one or more sensors can be positioned on a band 208 of the system 200, such that the body 216 is separate from the sensor module that includes the heat source 210 and the one or more sensors.

In some implementations, such as shown in FIG. 2C, the heat source 211 and/or one or more sensors 213 of the system or device 201 can be distributed between the band 208, body 216, and sensor module 209 depending on which sensors are incorporated into the system 201 and their specific requirements or parameters. In a still further implementation, the sensor module 209 can be positionable in an in-ear device (e.g., car lobe clip, car bud, hearing aid, etc.). The sensor module 209 can be configured to measure one or more parameters, depending on which sensors 213 are present, for example blood pressure, temperature, and/or oxygen saturation.

Further, the heat source 210 can be communicatively coupled to a hardware processor such that the hardware processor outputs a heating signal to the heat source 210 to activate the heat source to initiate a heating cycle. For example, a heating cycle can include receiving baseline temperature signals from a skin temperature sensor and an environmental temperature sensor (for example, an ambient temperature), determining the target temperature based on the baseline temperature signals, and determining whether the target temperature is below a maximum temperature value.

In some implementations, a target temperature can be equal to a baseline skin temperature as measured by the skin temperature sensor plus an offset, for example about 1 to about 20 degrees, about 1 to about 5 degrees, about 2 to about 10 degrees, about 2 to about 15 degrees, about 1 to about 10 degrees, about 5 to about 10 degrees, about 5 to about 15 degrees, about 8 to about 12 degrees, etc. In one implementation, the target temperature is equal to the baseline skin temperature as measured by the skin temperature sensor plus about 5 to about 15 degrees. In another implementation, the target temperature is equal to the baseline skin temperature as measured by the skin temperature sensor plus about 7 to about 13 degrees. In another implementation, the target temperature is equal to the baseline skin temperature as measured by the skin temperature sensor plus about 10 degrees. If the target temperature is greater than a maximum temperature value, the system pauses or delays until the baseline skin temperature drops below a minimum threshold or recalculates the target temperature so that it is less than the maximum temperature value. If the target temperature is less than a maximum temperature sensor, the system proceeds to activate the heat source to heat the skin surface to the target temperature.

In some implementation, the heat source cycles between the target temperature and a deactivated or off state or between the target temperature and a temperature that is lower than the target temperature but greater than the skin baseline temperature, for example to maintain the target temperature, hereinafter referred to as a dwell time.

In some implementations, a duration of a heating cycle and a target temperature are interconnected and based on user preference or user perception of heat on the skin surface or a vasodilation response of the user. For example, a higher target temperature can be used for a shorter time period or a lower target temperature can be used for a longer time period.

Further, the system or device 200, 201 can be configured to receive one or more user inputs related to a perceived heat sensation on the skin surface and/or to a sensitivity of a vasodilation response of the user. For example, a user can input that the target temperature felt too hot or too cold, for example via a user input element (e.g., button), such that the system responds by reducing the target temperature but elongating an amount of time that the skin is heated. Additionally, or alternatively, based on user preference, preset configurations (e.g., during manufacturing), or as a result of sensed data (e.g., based on sensor data), the heat source can reach the target temperature via one of a plurality of ramping functions, for example slow ramping, larger step functions, etc. Alternatively, the heat source can reach the target temperature through a plurality of micro-stimulations. Further, for example, a target temperature can be individualized for the user based on the sensitivity of the vasodilation response of the user.

Further, as shown in FIGS. 2A-2C, a system or device 200, 201 for detection of an anomalous biologic event further includes, for example, a skin temperature sensor 214 and a blood volume sensor 212. The blood volume sensor 212 can be integrated into a form factor such as the device or system 200, 201 that improves continuous anomalous cardiac event monitoring. The blood volume sensor 212 can measure parameters that can provide vasodilation response. Furthermore, the skin temperature sensor 214 can also be integrated into the system or device 200, 201. The skin temperature sensor 212 can positioned on the second surface 202 and configured to measure a temperature of the skin surface in contact with the heat source 210. Additionally, one or more sensors 213 can positioned on the sensor module 209 and configured to measure a temperature of the skin surface in contact with the heat source 211. The blood volume sensor (e.g., blood volume sensor 212 and/or one or more sensors 213) can be a photoplethysmography sensor or an impedance plethysmographic sensor. The blood volume sensor can employ light at 530 nm (green), 645 nm (red), 470 nm (blue) wavelength, or a combination thereof. Different wavelengths can be more appropriate for different applications, for example green (530 nm) light can be more accurate for heart rate measurements (e.g., heart rate variability, heart rate, etc.). In addition to, or alternatively, the blood volume sensor 212 and/or one or more sensors 213 can be further configured to measure one or more of: heart rate, heart rate variability, or oxygen saturation.

A system or device 200, 201 for detection of an anomalous biologic event can include an environmental temperature sensor configured to measure a temperature of the environment around the wearable system 200, 201. For example, an environmental temperature sensor, such as ambient temperature can be positioned on the first surface 204 of the body 216 of the wearable system, opposite the second surface 202 that includes the heat source 210. Alternatively, the system or device 200 can be communicatively coupled to an environmental temperature sensor on or in a remote computing device. For example, the remote computing device can include a laptop, a cellular device, a workstation, a server, a desktop computer, a personal digital assistant, a second wearable system or device, a netbook, or the like.

The skin temperature sensor and/or environmental temperature sensor can include a thermocouple, a resistance temperature detector, a thermistor, or an infrared temperature sensor. The type of temperature sensor selected can depend on error rate, coupling to skin surface efficiency, among other features.

In some implementations, the heat source 210 is positioned concentrically about any one or more sensors. For example, a blood volume sensor 212 (shown in FIGS. 2A-2B), a skin temperature sensor 214, or additional sensors, for example an EDA sensor, a potentiometric ion-selective electrode sensor, a colorimetric sensor, or an enzymatic amperometric sensor can be positioned such that the stimulation source, e.g., heat source 210, is positioned about, encircles, encapsulates, or is concentrically disposed about the sensor. Although, a location or position of the blood volume sensor 212 and the skin temperature sensor 214 that enables coupling to a skin surface is envisioned. The blood volume sensor 212 can be configured to measure one or both of a heart rate and a blood volume of the user.

A hardware processor (within the wearable system or communicatively coupled to the wearable system) communicatively coupled to the skin temperature sensor 214 and the environmental temperature sensor can be configured to perform a method comprising: receiving a first temperature signal using the skin temperature sensor and a second temperature signal using the environmental temperature sensor; and calculating a temperature differential between the skin temperature and the environment temperature. For example, if the temperature differential is below a set threshold, a difference between the target temperature and the maximum temperature value can be increased. In contrast, if the temperature differential is above a set threshold, a difference between the target temperature and the maximum temperature value can be reduced. The environmental temperature sensor can also be used in analysis of determining erroneous results, such as false positive indications of abnormalities. By comparing signals before and after stimulus and/or by comparing left versus right limb, externalities such ambient temperature response can be reduced in the analysis of abnormalities.

Further, the hardware processor can be coupled to the heat source 210 and the blood volume sensor 212. In some implementations, the system or device 200, 201 described above can enable non-invasive monitoring of vasodilation and/or vasoconstriction. Human body regulates stable equilibrium through the process of homeostasis. For example, if a stimulus is applied to a body of patient, one or more homeostatic processes will attempt to counteract the effect of stimulus. For example, with respect to an induced thermal stimulus that increases or decreases temperature at a tissue site, the body will attempt to reverse the temperature change through blood flow (vasodilation or vasocontraction). Accordingly, the system or device 200, 201 can induce and measure the vasodilatory response. As discussed above, stroke and other abnormalities can impair the vasodilatory response. Therefore, in some implementations, it can be advantageous to monitor the change in the vasodilatory response to determine abnormalities, such as stroke. A blood volume sensor, such as optical sensors, can enable monitoring of the blood flow and correspondingly the vasodilatory response. In some implementations, one or more temperature sensors (through a thermistor or optical radiation-based detectors) can also enable determination of the vasodilatory response by monitoring how quickly the temperature of the skin returns to equilibrium following the stimulus. In some examples, the vasodilatory response is correlated with a rate of change or slope in the measured parameter, such as blood volume parameters, temperature, and others discussed herein. In additional examples, the vasodilatory response can be correlated with a steepness of the rate of change. This can be calculated using a second derivative.

In some implementations, it can be advantageous to use a combination of a heat source 210 and the blood volume sensor 212 to improve cardiac monitoring. The heat source 210 and the blood volume sensors 212 can be integrated into a form factor that a user can wear for continuous monitoring. The measurements can be repeated non-invasively without significant discomfort to the patients. Furthermore, the response time between the application of heat and the change in blood volume is relatively small. This can enable a relatively fast determination of the anomalous biologic event. Therefore, it can be advantageous to integrate a heat source and a blood volume sensor in any wearable system disclosed herein to improve continuous cardiac monitoring. In some implementations, a Peltier cooler can be used as a thermal source instead of or in addition to the heat source 210.

Furthermore, in some implementations, the stimulus can be an electrical stimulus in addition to or instead of the thermal stimulus. For example, the system or device 200, 201 can include a plurality of electrodes for applying current and/or voltage to a circuit associated with the plurality of electrodes in order to measure particular signals (e.g., electrical activity) from a tissue site. Electrical activity can include bioimpedance for detecting high or low muscle tone, which can occur with hemiplegia. The system or device 200, 201 can include at least two electrodes. In some implementations, the system or device 200, 201 can include three electrodes. In some implementations, the system or device 200, 201 can include at least four electrodes. Furthermore, the system or device 200, 201 can also include six or more electrodes. The electrodes can be integrated on the system or device 200, 201 such that they are in contact with the skin tissue of the user.

As discussed above, an optical sensor, such as the blood volume sensor 212, can interrogate a target tissue to determine parameters that correlate with the vasodilatory response. Other sensors can also be used to extract parameters for determination of the vasodilatory response. For example, the system or device 200, 201 can use minimally invasive and/or invasive sensors to determine hemodynamic parameters, such as cardiac output, to provide an indication of the vasodilation response. The system or device 200, 201 can also include on or more electrical based sensors, such as bioimpedance sensors, EDA sensors, ECG sensors, EEG sensors, EMG sensors, and the like. Electrical sensors can enable measurement of hydration, skin conductance, and other electrical parameters that relate to hemodynamic function monitoring. Furthermore, the system or device 200, 201 can include one or more ultrasound sensors to obtain hemodynamic parameters. Temperature sensors can also enable determination of the vasodilation response. Accordingly, the system or device 200, 201 can include a combination of some or all of the sensors discussed above to extract one or more parameters that correlate with hemodynamic function or maintenance of homeostasis.

Patients are often monitored in neuro ICU after a stroke. This can be expensive as a human (e.g., a nurse) typically conducts periodic checks on the patient. Such human-based checks can also be subjective and/or inaccurately timed, thus missing conditions, symptoms, and/or events. Accordingly, the system or device 200, 201 can enable improved monitoring without requiring the patient to be in the neuro ICU and/or without requiring a caregiver to conduct periodic checks. While the system or device 200, 201 is described as a wearable system, in some examples, some or all of the components of the system 200 can be positioned in proximity to the user but not directly attached or worn by the user. For example, when a user needs to be monitored in a hospital environment, some or all of the components of the system or device 200, 201 can be positioned in proximity to the user's hospital bed. For example, the thermal stimulus source can include a laser.

In any of the implementations described herein, a wearable system or device 200, 201 for detecting anomalous biologic events can include one or more electrodermal activity sensors positioned on the second surface and/or a tensionable band of the system. For example, as shown in FIG. 2B, electrodermal sensors 224, 226 are positioned on the second surface 202 of the wearable system 200. Electrodermal sensors 224, 226 can be spaced apart from one another by distance 244, which equals about 5 mm to about 10 mm, about 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of each sensor. Further, electrodermal sensors 224, 226 can be spaced apart from the heat source by distance 246, which equals about 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of the sensor and a center point of the heat source.

In any of the implementations described herein, a wearable system or device 200, 201 for detecting anomalous biologic events can include one or more motion sensors (not shown) configured to measure a motion of a body portion to which the wearable system 200, 201 is coupled. For example, the one or more motion sensors can measure an acceleration in six or nine degrees of freedom. As described elsewhere herein, a wearable system or device 200, 201 for detecting stroke can, in combination with measuring a vasodilation response in response to application of heat, can measure asymmetrical movement or tremors of the right and left limbs. One or more motion sensors can be positioned anywhere on the wearable device 200. For example, in one implementations, a motion sensor is positioned in or on the first surface 204. In another implementation, a motion sensor is positioned in or on the second surface 202. In another implementation, a motion sensor is positioned in between the first and second surfaces 204, 202. In another implementation, a motion sensor is positioned on a sidewall of the body of the wearable device. In another implementation, a motion sensor is positioned adjacent to a vasodilation sensor or temperature sensor of the system, for example concentrically surrounded by the heat source.

The heat source (e.g., heat source 210) of the wearable system or device 200, 201 can be cooled in between heating cycles to ensure a return to baseline or substantially baseline of the vasodilation response of the skin surface in between heating cycles. As such, the heat source can be cooled by an airflow system (e.g., fan), a vacuum or vibrating mechanism configured to displace or pull or move environmental air across the heat source (e.g., solenoid and diaphragm, oscillating piezo element), etc. In one implementation, as shown in FIG. 2B, a wearable system or device 200, 201 for detecting an anomalous biologic event includes first 204 and second 202 surfaces that together define a cavity 206 therebetween to provide airflow between the first 204 and second 202 surfaces. The cavity 206 defined by the first 204 and second 202 surfaces can physically separates the heat source 210 from a hardware processor (not shown) positioned on or within the first surface 204. The hardware processor can include microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein. The cavity 206 can function to expose the heat source 210 to ambient or environmental or surrounding air to cool the heat source 210 to a temperature that approaches, substantially equals, or equals a temperature of the air in the environment or an ambient temperature. The cavity 206 can be an empty space, an interstitial space, a space that houses one or more components, etc. In some implementations, the cavity 206 formed by the first 204 and second 202 surfaces is open to ambient air or environmental air such that the sidewalls 205 that couple together the first 204 and the second 202 surfaces are opposite one another so that the cavity 206 is open to the environmental air on opposing sides, as shown in FIG. 2B. Alternatively, the sidewalls 205 are connected to one another and adjacent to one another so that the cavity is open to the environmental air on adjacent or connected sides.

For example, in some implementations, the cavity 206 defined by the first 204 and second 202 surfaces has sufficient volume to facilitate cooling of the heat source 210 in between heating cycles. Alternatively, or additionally, the cavity 206 can further include an airflow system, vacuum or vibrating mechanism, or other airflow mechanism to promote airflow through the cavity 206 to reduce a temperature or cool the heat source 210.

In some implementations of the wearable system or device 200, 201, the device includes 200 a port 220 for electrically coupling the device 200 to a power source, for example to charge a battery in the device 200. Additionally, or alternatively, port 220 electrically couples the wearable device 200 to an external or remote computing device (e.g., laptop, desktop, server, workstation, etc.) to download data from the device or upload system parameters or install updates to the wearable device. The wearable device can further include one or more user input elements 218 to power on and off the device; to input user specific reactions, features, or characteristics, to customize an interface or functionality of the user device, etc.

### Example Features

A multivariate stroke detection system 1000 can be configured to extract one or more bilateral and/or systemic features of a physiological condition of a monitored person or patient based at least in part on one or more physiological signals and/or other patient data. As will be discussed herein, various extracted features can be analyzed by a controller of a system 1000 in order to determine one or more neurological conditions or factors associated with a patient, such as the presence or likelihood of a stroke having occurred, presently occurring or likely to occur, a type and/or location of that stroke, a presence, degree or severity of neurological decline, the like or a combination thereof.

Disclosed herein are some non-limiting examples of bilateral and systemic features that can be extracted by a controller of a system 1000. A systemic feature can include, but is not necessarily limited to, a feature associated with a physiological condition as detected in a physiological signal or set of physiological signals indicative of a medical anomaly that can be detected in the physiological system of the person 100 as a whole, such as fever, increased blood pressure, changes in heart rate variability and others. A bilateral feature can include, but is not necessarily limited to, a feature of a physiological condition as detected in a physiological signal or set of physiological signals indicative of a medical anomaly that can be detected differently at different lateral sides, such as left lateral 101A and right lateral 101B of the body, such as asymmetry of vasodilation response, skin temperature, sweating, muscle activity, and others.

A feature can be based on inter-individual or intra-individual tracking and/or information. An inter-individual feature can be a feature based on a comparison of healthy and anomalous patient populations, such as a population who has not experienced a stroke as compared to a population who has experienced a stroke. For example, an inter-individual feature can relate to a feature and/or physiological condition recognized in population studies and/or theorized across multiple individuals to be associated with an elevated risk of an anomalous medical event. An intra-individual feature can be a feature based on a change in physiological condition of an individual over time, such as a comparison of historical trends or values of a physiological variable and/or parameter to current trends or values. For example, an intra-individual feature can relate to a sudden change in a value or trend that can be associated with an elevated risk of an anomalous medical event even where an individual value or trend across different individuals may or may not be associated with an elevated risk.

It is of note that certain features described herein can be individually strongly correlated with a physiological condition of a person in relation to an anomalous medical event, such as a stroke. However, other features can be correlated with a physiological condition when paired with or compared to different features. For example, bilateral asymmetry in muscle activity on its own can be highly correlated with the onset of a stroke whereas heart rate variability (HRV) on its own may not be as strongly correlated with the onset of a stroke without comparison or pairing of HRV with a secondary feature, such as bilateral asymmetry in skin temperature response to a heat stimulus. Accordingly, different features can be associated with different confidence values or weights associated with risk of an anomalous medical event, such as a stroke.

Advantageously, one or more features and/or physiological signals can be combined to identify a group of features that can be more strongly associated with an acute risk in addition or in the alternative to using a single feature and/or physiological signal. For example, where multiple features indicate a high risk of stroke, the system 1000 can identify a high confidence value for a calculated risk as compared to a single feature indicating a high risk of stroke.

Additionally, one or more features and/or physiological signals can be excluded and/or selected for determination of an anomalous medical event in accordance with other contextual data associated with the person. Additionally or alternatively, one or more features and/or physiological signals can be assigned a confidence value in accordance with other contextual data associated with the person. For example, a person can have a heart condition regardless of a stroke that can give a false positive for stroke and thus, heart related parameters or features can be of less utility in identifying a stroke in such a patient. Accordingly, in some examples, one or more features can be selected, excluded, or assigned a confidence measure or weight, based on patient specific medical history, demographic information, or other patient specific data.

Non-limiting examples of features that can be utilized by a system 1000 are shown in Tables 1 and 2 and discussed herein.

### Table 1

Showing non-limiting examples of bilateral features or features that can be identified in a variety of measured physiological parameter signals or signal types.

| Physiological Parameter | Feature - Bilateral Difference |
|---|---|
| Skin Temperature (e.g., skin | Absolute value of asymmetry in Rate of Change after |
| temperature sensor, IR | Stimulus |
| thermometer) | Absolute value of asymmetry in ambient temperature vs. |
| | Skin temperature |
| | Baseline temperature asymmetry |
| | Time to Threshold temperature drop level |
| | Asymmetry in slope of temperature change with time as |
| | a response to stimulus |
| | Peak Temperature Asymmetry |
| Sweating (e.g., EDA signal, | Absolute Conductivity |
| GSR signal) | Count of events of sweat gland firing |
| | Asymmetry of tonic sweat response to thermal stimulus |
| Muscle Activity (e.g., EMG | Measure of muscle recruitment in response to |
| signal) | contraction instruction |
| | Asymmetry in power spectral density (PSD) |
| | distributions |
| | In general, preferably after defined trigger or action task |
| | Median frequency of power spectral density |
| | Mean frequency of power spectral density |
| | Maximum frequency of power spectral density |
| | Coefficient of variance of time-domain EMG signal |
| | Integration of time-domain signal |
| | Envelope of time-domain signal -> current amplitude of |
| | the EMG signal |
| | Interquartile range (IQR) of power spectral density |
| | Interquartile range (IQR) of time-domain signal |
| | Variance of time-domain signal |
| | Time since EMG amplitude was above resting/no |
| | movement threshold |
| | Asymmetry in time since EMG amplitude was above |
| | resting/no movement threshold |
| Vasodilation (e.g., PPG signal) | % Vasodilation increase asymmetry (cause by equal |
| | heat stimulus on both wrists) |
| | % Vasodilation decrease asymmetry |
| | Time to reach vasodilation trough |
| | Time to reach vasodilation peak |
| | Time delays to reach % Vasodilation increases |
| | Time delays to reach % Vasodilation decreases |
| | Slope at specific time delays |
| | Latency to start vasodilating after thermal stimulus |
| | Asymmetry in Percentage differences of vaso |

### Table 2

Showing non-limiting examples of systemic features or features that can be identified in a variety of measured physiological parameter signals or signal types.

| Physiological Parameter | Feature - Systemic |
|---|---|
| Blood Oxygenation (e.g., PPG) | Static spO2 |
| | Evolution of spO2 over time |
| Blood Pressure (e.g., Cuff or | Evolution of diastolic blood pressure (DBP) over time |
| PPG) | Evolution of systolic blood pressure (SBP) over time |
| | PPG Pulse pressure |
| | Pulse Wave Analysis features |
| Heart Rate (e.g., ECG signal, | Resting heart rate evolution |
| PPG signal) | Time HR takes to return to baseline after activity |
| Heart Rate Variability (e.g., | TINN (Triangular interpolation of N-N interval |
| | histogram) |
| EKG signal, PPG signal) | PNN50 (Percentage N-N intervals >50 msec) |
| | RMS of standard deviation of N-N intervals |
| | Triangular Index |
| | HRV Frequency domain features |
| | Total power LF/HF |
| | SDNN (Std Dev of N-N Intervals) |
| Respiratory Rhythm (e.g., PPG | Respiration pattern |
| signal, chest sensor, | Apnea Hypopnea Index |
| microphone) | |
| Shivering (e.g., IMU, camera) | Presence, in general |
| Skin Temperature (e.g., skin | Time to peak temperature |
| temperature sensor, IR | Slope at specific time delays |
| thermometer) | |
| Sweating (e.g., EDA signal, | Absolute Conductivity |
| GSR signal) | Count of events of sweat gland firing |

In some examples, a feature can be related to blood oxygenation, such as SpO2. A blood oxygenation feature can be systemic and can include, for example, a threshold or feature associated with a value or trend of blood oxygenation, such as SpO2. The blood oxygenation can be measured from one or more optical sensors, such as a photoplethysmography or PPG sensor.

In some examples, a feature can be related to blood pressure. A blood pressure feature can be systemic and can include, for example, a threshold or feature associated with a value or trend of a diastolic blood pressure (DBP) and/or systolic blood pressure (SBP) over time. A sudden increase in DBP or SBP can be an indicator of increased intracerebral pressure, which can occur with stroke onset. Additionally or alternatively, a blood pressure feature can include a threshold or feature associated with a PPG pulse pressure and/or pulse wave analysis feature. PPG pulse wave AC amplitude and/or pulse wave features are tied to blood pressure and blood flow metrics. Thus, features of these signals and/or values can be utilized as an indicator of stroke onset. The blood pressure and/or pulse wave features can be measured from data obtained by one or more sensors, such as one or more optical sensors (e.g., photoplethysmography or PPG sensor) and/or a blood pressure cuff.

In some examples, a feature can be related to heart rate. A heart rate feature can be systemic and can include, for example, a threshold or feature associated with a resting heart rate of a person, trends associated with a resting heart rate of a person, and the time it takes a person's heart rate to return to a baseline value. Heart rate features can change during a stroke due, for example, to sympathetic overdrive that occurs during a stroke and the inflammation response. Thus, features of these signals and/or values can be utilized as an indicator of stroke onset. The heart rate can be measured from data obtained by one or more sensors, such as an ECG/EKG or PPG sensor.

In some examples, a feature can be related to heart rate variability. A heart rate variability feature can be systemic and can include, for example, a threshold or feature associated with heart rhythm features, such as PNN50 (or percentage of N-N intervals greater than 50 milliseconds, which is typically lower in stroke subjects compared to non-stroke subjects), RMS of standard deviation of N-N intervals (which is typically lower in stroke subjects compared to non-stroke subjects), TINN (or triangular interpolation of N-N interval, which is typically lower in stroke subjects compared to non-stroke subjects), HRV frequency domain features (which typically shows more energy at lower frequencies for stroke subjects compared to non-stroke subjects), total power LF/HF ratio (which typically shows a lower LF/HF ratio for stroke subjects compared to non-stroke subjects), and SDNN (or standard deviation of N-N intervals, which is typically lower in stroke subjects compared to non-stroke subjects). The heart rate variability can be measured from data obtained by one or more sensors, such as an ECG/EKG, or PPG sensor.

In some examples, a heart rate variability (HRV) can be continuously monitored using wearable technology. Without continuous monitoring, acute neurological changes can go undetected in both community and inpatient settings. For example, despite frequent neurochecks, only 45 percent of neurological deterioration can be captured during or as a result of a scheduled assessment. Because certain autonomic changes as a result of acute ischemic stroke (AIS) be detected non-invasively, monitoring of one or more of these changes, such as via a wearable device that can be worn regularly and/or continuously by a person, acute brain injuries can be detected in real time and/or more quickly than with standard neurochecks. Example autonomic changes that can result from an AIS can include changes in cardiac function, blood pressure and/or skin temperature. For example, in stroke patients, heart rate can increase and HRV can decrease compared with healthy persons. This HRV reduction as a result of AIS can occur due to autonomic dysfunction resulting in sympathetic overdrive.

Further, because accurate and timely stroke diagnosis within the first 4.5 hours after symptom onset is paramount when evaluating treatment options, such as intravenous thrombolytics (IVT), real time detection of neurological damage can be helpful in improving outcomes. While clinical examination and CT brain completion can be useful in this process, biometric could potentially be used by both patients and providers to aid in the recognition for diagnosis of stroke. In some examples, the biometric heart rate variability (HRV) can be used as a surrogate for autonomic nervous system function, with loss of HRV associated with autonomic dysfunction. Thus, reduction in HRV seen in the hyperacute period following symptom onset (for example, the 4.5 hours after symptom onset) can be used as a feature and/or biomarker of stroke.

In some examples, a feature can be related to respiratory rhythm. A respiratory rhythm feature can be systemic and can include, for example, a threshold or feature associated with a respiration pattern and an apnea-hypopnea index. Respiration rhythm features can change during a stroke due, for example, to unstable oscillations in respiration in response to oxygen demand. Thus, features of irregular breathing can be utilized as an indicator of stroke onset. The respiratory rhythm features can be measured from data obtained by one or more sensors, such as a chest or wrist sensor or PPG sensor.

In some examples, a feature can be related to involuntary physical movement of a person, such as shivering. A shivering feature can be systemic and can include, for example, a threshold or feature associated with shivering movement of a person, which can be associated with stroke onset. Involuntary (or voluntary) physical movement can be measured from data obtained by one or more sensors, such as an IMU or inertial measurement unit coupled to a person.

In some examples, a feature can be related to skin temperature. A skin temperature feature can be systemic or bilateral and can include, for example, a threshold or feature associated with a value or trend of an absolute value of bilateral asymmetry in a rate of change of temperature of the skin after a heat stimulus has been applied (where stroke subjects are likely to have a larger asymmetry compared to non-stroke subjects), an absolute value of asymmetry in ambient temperature vs skin (it is expected that stroke subjects stay warmer than non-stroke subjects after a heat stimulus is applied), a baseline temperature asymmetry (where stroke subjects present a larger asymmetry compared to non-stroke subjects), time to peak temperature after a heat stimulus is applied, a time to drop to a determined temperature after heat stimulus is applied, skin temperature slope asymmetries (where noticeable slope asymmetry differences can be observed in early stages of heating cycles and stroke subjects tend to have higher absolute slope asymmetries), peak temperature asymmetry (where higher peak temperature asymmetries can be associated with stroke onset). Thus, features of these signals and/or values can be utilized as an indicator of stroke onset. The skin temperature features can be measured from data obtained by one or more sensors, such as one or more optical sensors (e.g., IR sensor) and/or a contact temperature sensor.

In some examples, a feature can be related to a measured temperature, such as a wrist temperature, during a sleep cycle. Stoke is a non-traumatic injury to the brain that carries a recovery burden to survivors. In a neurocritical care unit (NCCU), patients are woken frequently to assess neurological state and identify neurological decline. However, sleep can be important for restorative activity of the body and brain. Despite optimizing factors to promote sleep in the NCCU, sleep remains sparse, which can confound these frequent neurological assessments. Advantageously, measuring continuous skin temperature at the wrist can help clinicians to better assess neurological activity of a patient with fewer sleep interruptions. For example, use of continuous skin temperature measurements can monitor a thermoregulatory status of a patient during sleep that can be altered as a result of a stroke event and contribute to improved sleep quality during stroke recovery.

For example, a feature can involve a comparison of a person's core body temperature and/or wrist temperature over a nighttime period with healthy and/or stroke survivor patient data. In healthy persons, core body temperature can drop by approximately 1 degree Celsius during a nighttime period compared with a daytime period (such as can be defined and/or in relation to a person's circadian rhythm). Further, wrist temperature of healthy subjects can have an increase of 3 to 7 degrees Celsius with the onset of sleep and remains elevated throughout the night and/or a sleep period. In stroke survivors, a wrist skin temperature can be more variable, such as up to 6 degrees Celsius or more variation throughout the night and/or sleep period. This variation, however, can be different and/or dependent on whether or not a stroke survivor sleeps during the night and/or sleep period. For example, a stroke survivor who does not have consistent sleep can have a variation in wrist temperature and a stroke survivor who has consistent sleep during the sleep period can have a consistent skin temperature (e.g., at approximately 37 degrees Celsius). Since wrist temperature (or other peripheral temperature measurement) can correlate with core and/or brain temperature, these differences in wrist temperature can be used as a feature such as referenced herein. For example, in healthy persons, a rise in peripheral temperature can indicate a drop in core and/or brain temperature to allow for the person to go through sleep cycles while this may not be observed in stroke survivor patients. This loss of diurnal peripheral skin temperature rise in stroke survivors suggests a lack of core temperature drop and expected sleep onset. This suggests that wrist skin temperature response can be a useful feature in and of itself (or in combination with other feature(s)) to understand and balance the relationship between the need for frequent neurochecks on a patient and allowing for sleep as a recovery process after neurological injury.

In some examples, a feature can be related to sweating. A sweating feature can be systemic or bilateral and can include, for example, a threshold or feature associated with a value or trend associated with the presence of sweating as detected by, for example, an electrodermal activity sensor (EDA sensor) or galvanic skin response sensor (GSR sensor) on the skin of the user, such as at the wrist. Non limiting examples can include but are not limited to absolute conductivity, a count of events of sweat gland firing, asymmetry of tonic sweat response to thermal stimulus.

In some examples, a feature can be related to vasodilation. A vasodilation feature can be bilateral and can include, for example, latency to start vasodilating after thermal stimulus, asymmetry in percentage differences of vasodilation, percentage vasodilation increase or decrease in asymmetry (such as caused by application or removal of equal heat stimulus to both right and left lateral sides of the body), time to reach vasodilation trough or peak, time delays to reach a percentage vasodilation increase or decrease, and a slope of vasodilation response at specific time delays. Vasodilation response can be measured, for example, based on features in an optical sensor, such as a PPG sensor or other sensor types. The vasodilation sensor can be located at each wrist or at other parallel bilateral locations of the body.

In some examples, a feature can be related to muscle activity. A muscle activity feature can be bilateral and associated with a response to stimulus or verified with confirmatory testing. Muscle activity can be measured via, for example, an EMG sensor at the wrists or at other parallel bilateral locations of the body. Examples of muscle activity features can include, but are not limited to, a measure of muscle recruitment in response to contraction instructions, asymmetry in power spectral density (PSD) distributions in an EMG signal, muscle movement in general or lack thereof and/or asymmetry in muscle movement or lack thereof, such as in response to contraction instructions, median, mean, or maximum frequency of power spectral density, a coefficient of variance of time-domain EMG signals (a reduced variance is associated with stroke), integration of time-domain EMG signals, amplitude of an envelope of time-domain EMG signals, interquartile range (IQR) of power spectral density or time-domain signal, variance of time-domain signal, time since an EMG amplitude was above resting or a no movement threshold, and an asymmetry in time since an EMG amplitude was above resting or a no movement threshold.

It is of note that other features and/or combinations of features can be utilized and the examples described herein are meant to be non-limiting. The features can be determined based on any number of factors, such as population studies, expected physiological responses to stimulus or instructions (e.g., instructions to perform a movement, such as clenching both fists), physiological parameter trends identified in the patient or other populations, the like or a combination thereof.

### Example Feature Mapping

One or more features, such as described herein, can be utilized as part of a risk classification for an anomalous medical event, such as a stroke and/or utilized to identify one or more parameters and/or characteristics of such an event. For example, the system 1000 can utilize a single feature strongly correlated with a stroke as an indicator of an anomalous medical event, such as a stroke. In further examples, the system 1000 can utilize a mapping of multiple features into a multi-dimensional (for example, two or three dimensional) space to help analyze and classify the data for identification of an anomalous medical event. The utilization of multiple features can allow for more accurate classification of medical events and, in some instances, allow for generation of a more nuanced identification of event classes, such as what type and/or where a stroke can have occurred physiologically.

It is of note that utilization of multiple feature can help filter false positives from stroke features that are similarly occurring in non-stroke individuals, such as low Heart Rate Variability. Additionally, for an anomalous medical event, such as a stroke event, it is of note that different types of features and/or metrics can vary based on stroke type or location. For example, an individual can have ipsilaterally warmer skin temperature asymmetry, and another individual can have a contralateral warmer skin temperature asymmetry, based on the location of the stroke. Thus, multiple features can be utilized to account for false negatives.

As will be discussed herein, event classification can be based on feature mapping utilizing at least some of the plurality of features disclosed herein. However, these features and/or particular mappings are non-limiting and different mappings can be utilized than disclosed in the non-limiting examples discussed herein. In some examples, multiple mapping of different and/or overlapping features can be utilized for event classification. For example, a system 1000 can make a plurality of classifications based on a plurality of mappings and/or individual features. The plurality of classifications can be made in series or in parallel.

The system 1000 can be configured to utilize features selected based on a static or dynamic set of features. In some examples, the system 1000 can determine which features to utilize based on a predetermined set of parameters based on, for example experimental or clinical data. Additionally or alternatively, the system 1000 can determine which features to utilize based on a hardware configurations, such as sensors available, power management, hardware availability, other parameters, the like or a combination thereof. Additionally or alternatively, the system 1000 can determine which features to utilize based on user indicated parameters, such as patient specific data, clinician preference, the like or a combination thereof. Additionally or alternatively, the system 1000 can determine which features to utilize based on the collected patient data itself, such as the quality of the sensor signal, features identified in the signals, the like or a combination thereof.

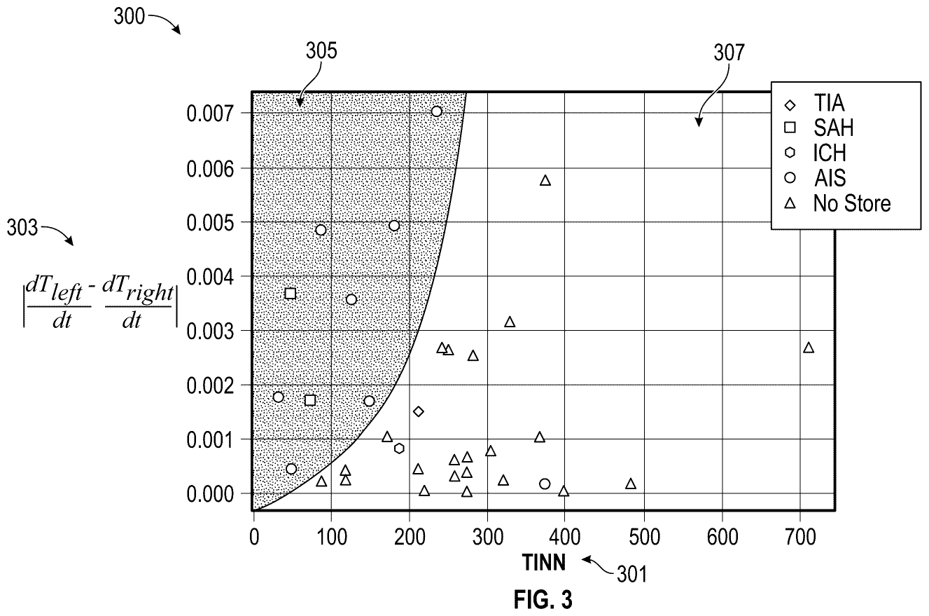

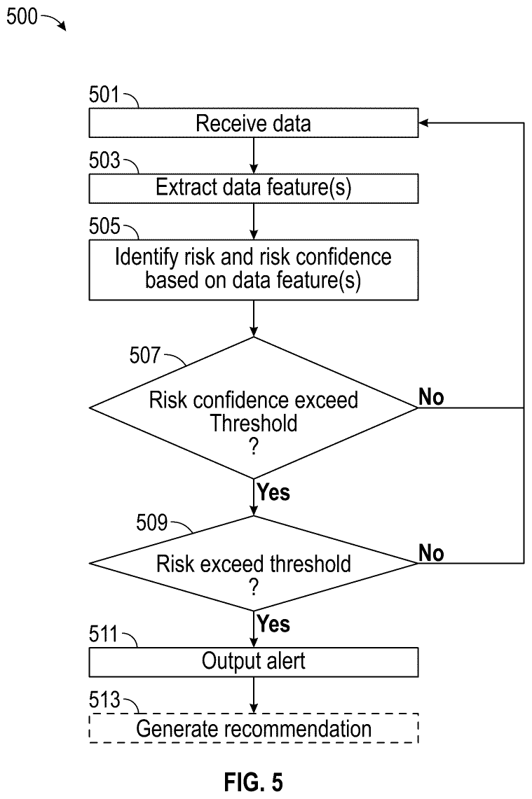

FIG. 3 illustrate an example feature mapping that can be utilized as part of a risk classification by a system 1000 as a basis of comparison for data of monitored persons. For example, a system 1000 can compare data from a monitored person or patient to the illustrated mappings in to help identify and/or classify a potential event experienced by the monitored person or patient. The feature mapping examples disclosed herein relate to intra-individual feature mapping such that each data point on the mappings relate to a different individual either having had a stroke event or a non-stroke event. The stroke events, in some cases, are classified by stroke type. Further details of example processes that can utilize such mappings are discussed herein with reference to, for example, FIG. 5. As shown in FIG. 3, the data values corresponding to the plurality of individuals can be sorted between a shaded area 305 and area 307 as further described below. A boundary between the shaded area 305 and area 307 can serves as a decision threshold that separates the shaded area 305 (high-risk AIS patients) from area 307 (lower-risk patients). This boundary line can represent the point at which a classifier determines the likelihood of a patient experiencing a true stroke based on their measured values. If a patient's data point falls near or on the boundary, their risk level may be less certain, requiring further evaluation. The classifier uses this boundary to systematically differentiate between AIS and non-AIS cases, ensuring more accurate and reliable stroke risk assessment. The classifier can also be a machine learning based classifier. The trends described herein can be used as inputs to train a machine learning based classifier. Certain variables, such as metrics of heart rate variability, were identified by inventors to result in an improved classifier for stroke detection. Furthermore, asymmetry between a left limb and a right limb in response to stimulus may further be an input for the classifier.

FIG. 3 illustrates an example feature mapping 300 of data values for bilateral asymmetry in thermoregulation 301 as a function of measure of heart rate variability 303 for a plurality of individuals having different medical events, such as a transient ischemic attack (TIA or a “mini stroke”), subarachnoid hemorrhage (SAH), intracerebral hemorrhage (ICH or a “hemorrhagic stroke”), arterial ischemic stroke (AIS or a “true stroke”), and no stroke.

With reference to FIG. 3, the measure of bilateral asymmetry of thermoregulation 301 can include an absolute value of the difference of the slope of skin temperature with respect to time as a result of the application of a heat stimulus to a left and a right lateral side of the body of the user. The measure of heart rate variability 303 can include the triangular interpolation of N-N intervals in an ECG signal or (TINN). TINN is a measure of heart rate variability (HRV) that quantifies the baseline width of the RR interval histogram on an electrocardiogram (ECG). TINN represents the distribution of RR intervals and can be used to assess autonomic regulation. A wider TINN value indicates greater variability in RR intervals, while a narrower TINN value suggests reduced variability, which may indicate autonomic dysfunction or cardiovascular impairment. For example, a larger TINN is indicative of greater variation in N-N intervals of a heart rhythm and heart rate variability (HRV) is the fluctuation in the time intervals between adjacent heartbeats. TINN can also be used as a classification feature for assessing physiological conditions and stroke risk.

Heart rate variability (HRV) is comprised of changes in the time intervals between consecutive heartbeats called interbeat intervals (IBIs). HRV can be an indicator of neurocardiac function, arising from interactions between the heart, brain, and autonomic nervous system (ANS). HRV can help regulate vital physiological processes such as blood pressure, gas exchange, and vascular tone, allowing the body to adapt. A healthy heart does not beat with perfect regularity but instead exhibits complex, non-linear fluctuations that support resilience and adaptability. However, deviations from this balance-either excessive or diminished HRV—can signal underlying health issues. It is of note that people who have had a stroke have a more steady heartbeat and thus, a lower HRV due to sympathetic overdrive as a result of a stroke event. In contrast, a healthy individual has a better paring of sympathetic and parasympathetic activity, which both speeds up a slows down heart rate in turn, resulting in a higher heart rate variability. However, HRV alone may not necessarily be a strong predictive indicator of stroke at least in part due to other potential reasons for low HRV, such as poor cardiovascular health. Thus, while HRV can serve as a good biomarker of stroke (such as a change to HRV within a window after a stroke event), utilization of a secondary metric to identify a correlation of HRV and stroke can help ensure a more accurate identification of stroke, especially after an initial period following a stroke event.

The selection of data for skin temperature can be a window of data over a period of time, such as 10, 20, 30, 40, 50, or 60 seconds or more. The period of time can be related to an amount of time after a stimulus has started (e.g., after a heater is turned on), after a stimulus has reached a threshold (e.g., after a heater has reached a maximum power), after a stimulus has turned off, the like or other metric related to stimulus application. In other examples, the period of time can relate to an amount of time during which the data is of sufficient quality to generate a reasonable result. For example, the data can be processed, filtered and/or windowed to generate a Signal to Noise Ratio (SNR) satisfying a threshold quality to map. The period of time can be some combination of metrics, such as a period of time during which the stimulus is applied and during which the SNR is greater than a threshold. Other periods or windows can additionally or alternatively be utilized based on other factors impacting the collection and/or analysis of data.

Mapped feature types may or may not be collected within the same or even overlapping windows of data collection. In some examples, mapped feature types can be collected in the same time period. In some examples, mapped feature types can be collected in different time periods. For example, a heart rate variability metric, such as TINN can be collected up to 30 minutes or greater after a skin temperature metric is collected. However, in some cases, feature types can be correlated in time to include the same or overlapping windows of data collection. Whether mapped feature types are collected in the same window may or may not be based on likelihood of physiological changes within that window. For example, a heart rate variability metric can be less likely to change within a 30 minute period than a muscle activity metric and thus, the data collection windows may not need to overlap.

As illustrated, the shaded area 305 of the plot is associated with a grouping of AIS type stroke patients in comparison to the area 307 of the plot. The shaded area 305 can serve as a classification boundary to help determine whether a patient is a high risk for AID. The shaded area 305 area can be identified based on specific measured values, such as asymmetry in thermoregulation, which can serve as indicators of stroke risk. When a new patient's data falls within this region, it can suggest a higher likelihood of experiencing a true stroke, making it a crucial factor in risk assessment. In contrast, area 307 can represent a region where data points are less associated with AIS, indicating a lower probability that a patient is experiencing a true stroke. Patients whose measurements align with area 307 can exhibit stroke-like symptoms due to other conditions, but they are statistically less likely to have AIS. These distinct areas can help form a decision boundary for classification, allowing models to differentiate between high-risk and lower-risk patients. Thus, measured data of a patient plotted to fall within the identified area 305 of the scatter plot 300 can indicate a higher degree of risk of that patient experiencing a “true stroke” or AIS than were the measured data to fall within the area 307 of the scatter plot 300. Accordingly, a feature for acute stroke risk can be identified as whether a measure value falls within that shaded area 305 of the plot 300, such as through comparison of the measure of asymmetry of thermoregulation to a threshold associated with a TINN value associated with the measured person.

In features with a strong correlation, the grouping of stroke types can be strong, such as illustrated in FIG. 3. In some cases, different types of strokes and/or where they occur (e.g. cortical or subcortical) can be identified based on a feature mapping, such as FIG. 3. For example, a location of a data point in a particular area of the mapping could indicate an AIS, ICH, or SAH type stroke event. In some cases, a system 1000 can utilize multiple features and/or multiple mappings to determine a stroke and/or stroke type.

Accordingly, as discussed elsewhere herein, a system can utilize this population data to test a user for stroke risk. For example, the system can apply a heat stimulus to a left and right lateral side to compare a slope of temperature change across each and compare that value to a threshold asymmetry based on the TINN 303 determined using an ECG signal in order to identify an acute risk of a true stroke.

Other example feature mappings can include, but are not limited to, temperature slope during heater application as a function of a heart rate variability metric (such as can be measured by, for example, one or more EMG features), an asymmetry of temperature decrease after cessation of a heat stimulus as a function of a heart rate variability metric, an asymmetry in baseline temperature as a function of a heart rate variability metric, or others.

### Example Multivariate Assessment Processing

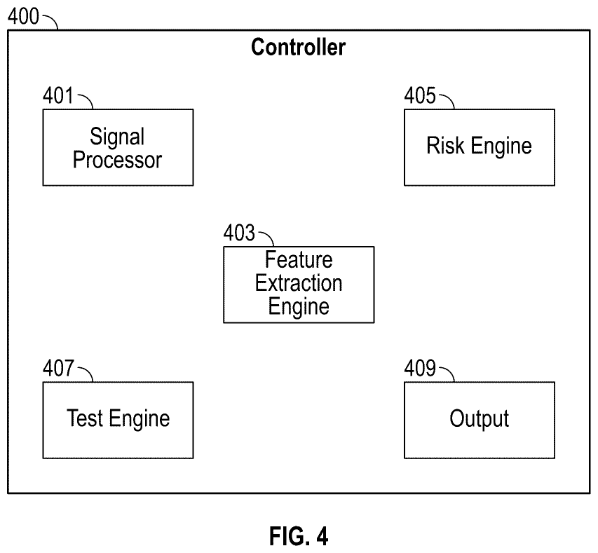

FIG. 4 illustrates an example controller 400 which can be part of a multivariate stroke detection system 1000. The controller 400 can be one or more hardware processors associated with one or more local or remote computing devices, such as one or more physiological sensors 103, one or more local computing devices 105, or network devices 107 disclosed with reference to FIG. 1. The controller 400 can be configured to perform one or more processes relating to detection and monitoring of a medical anomaly, such as a stroke. For example, the controller 400 can include, but is not limited to at least one of a signal processing engine 401, feature extraction engine 403, risk engine 405, test engine 407, and output engine 409.

A signal processing engine 401 can include one or more processes for receiving and/or processing signals from one or more physiological sensors and/or monitoring devices, such as one or more physiological sensors 103. The signal processing engine 401 can be configured to process raw signals and/or received physiological parameters in order to facilitate determination of a risk of an anomalous medical event. The signal processing engine 401 can be configured to process signals from one or more sensor types, such as an EEG, ECG, EKG, PPG, EDA, GSR, temperature sensor, respiratory sensor, microphone, camera, other sensor types or a combination thereof. In some examples, the signal processing engine 401 can be configured to process a signal for feature extraction, such as by transforming a signal into a time-domain or frequency-domain, filtering a signal, performing noise cancellation, windowing a signal, or other pre-processing.

As referenced above, signals may or may not be collected and/or windowed within the same or even overlapping windows of data collection. In some examples, signals can be collected within the same time period. In some examples, signals can be collected within different time periods. For example, a heart rate variability metric, such as TINN can be collected up to 30 minutes or greater after a skin temperature metric is collected. Whether signals are collected in the same period and/or windowed within the same or overlapping period may or may not be based on likelihood of physiological changes within that window. For example, a heart rate variability metric can be less likely to change within a 30 minute period than a muscle activity metric and thus, the data collection windows may not need to overlap.

An extraction engine 403 can include one or more processes for extracting physiological data, such as physiological parameters or other data associated with physiological monitoring or a physiological condition of a patient from sensor or other input data associated with the patient. The extraction engine 403 can be configured to extract one or more features of the data in order to compare those features to certain known features associated with an anomalous medical event, such as identified elsewhere herein. For example, the one or more features can include systemic or bilateral features of sensor data, such as a degree of asymmetry of vasodilation in response to an applied heat stimulus at left and right bilateral locations of the body, which can both help with classification. The extraction engine 403 can be configured to extract one or more features from a single sensor type, multiple sensor types, other inputs, or a combination thereof.

A risk engine 405 can include one or more processes for determining a risk associated with an anomalous medical event, such as a stroke. The risk can be a quantitative or qualitative measure of the probability of an anomalous medical event having occurred, presently occurring, or likely to occur in the near future. The risk can be, for example, a percentage likelihood of the anomalous medical event having occurred, presently occurring, or likely to occur in the near future. The risk can be, for example, a binary determination of whether or not an anomalous medical event has occurred, is presently occurring, or is likely to occur in the near future or not. The risk can include, in some examples, a classification of an anomalous medical event, such as a type of stroke and/or where a stroke is likely to have or is presently occurring, such as cortical or subcortical.

The risk engine 405 can determine at least one risk based on one or more inputs, such as the features extracted by an extraction engine 403. The at least one risk can be an individual risk associated with an individual input or feature, a plurality of risks associated with a plurality of inputs or features, a total risk based on a combination of inputs or features, or a combination thereof. In some examples, a risk engine 405 can be configured to generate a plurality of risks and/or associated metrics based on one or more extracted features. The plurality of risks and/or associated metrics can, for example, be calculated independently, as part of a set of interdependent processes, in parallel, or in series. For example, a risk engine 405 can be configured to separately calculate a plurality of risks and/or associated metrics based on a plurality of different sets of feature types. A total risk can be a weighted combination of the plurality of calculated risks, a subset of the calculated risks, an individual risk selected from a plurality of calculated risks, or another value. In other examples, the risk engine 405 can be configured to perform a single risk calculation based on a single set of extracted features. In some examples, an output of the risk engine 405 can include individually calculated risks and/or associated metrics and/or an overall risk.

The risk engine 405 can be configured to additionally determine a confidence measure associated with a calculated risk. The risk engine 405 can be configured to generate an output of an overall risk based at least in part on the confidence measure. For example, the risk engine 405 can be configured to arbitrate individual risks based on a weighting of extracted features. The weights can be predetermined and/or dynamically updated based on, for example, a confidence measure of a calculated risk. A confidence measure can be associated with a strength of association of a feature or feature with the likelihood of stroke, a quality or validity of a signal, the like or a combination thereof.

The risk engine 405 can be configured to determine a risk based at least in part on a predetermined feature or feature threshold that is present or passed, respectively, in a set of input features. For example, a threshold degree of asymmetry in muscle activity, a threshold degree of asymmetry in thermoresponsiveness, a threshold change in blood pressure, or other feature thresholds such as referenced elsewhere herein can be utilized by the risk engine 405 to determine a risk of an anomalous event. In some examples, a feature threshold can be determined or set for a monitored person individually based on, for example, patient history and/or patient demographics. In some examples, a feature threshold can be determined based on clinical criteria not dependent on patient specific information. In some examples, a feature threshold can be updated or changed based on manual inputs or adjustments. In some examples, a feature threshold can be updated or changed based on an automated process. For example, the system can learn patient baselines, such as through machine learning techniques, and/or automatically adjust feature thresholds based on the learned patient baselines. In some examples, a feature threshold can be determined based on other inputs or factors.

Additionally or alternatively, the risk engine 405 can be configured to determine a risk based on a mapping of collected data to a predetermined mapping of features, such as discussed above with reference to FIG. 3. For example, the risk engine 405 can be configured to identify a risk associated with a stroke event and/or classify a stroke event based on the data location on a feature map determined using predetermined clinical data associated with a set of stroke and non-stroke patients that may or may not include the presently monitored patient.

Furthermore, the risk engine 405 can be configured to generate a classifier based on one or more features of the extracted set of features. For example, the risk engine 405 can be configured to identify a classifier associated with a stroke event based on the data location on a feature map determined using predetermined clinical data associated with a set of stroke and non-stroke patients that may or may not include the presently monitored patient. The classifier can be operable to categorize patient data into different risk levels associated with acute ischemic stroke (AIS). The classifier can utilize decision boundaries to distinguish between varying degrees of stroke risk based on predefined thresholds. For example, the one or more features of the extracted set of features can comprise a metric corresponding to heart rate variability (HRV). The one or more features can also include the other various metrics contained herein such as skin temperature, blood pressure, or other feature thresholds such as referenced elsewhere herein can be utilized by the risk engine 405 to generate classifier.

A test engine 407 can include one or more processes for testing a user for a feature by determining a feature or feature of an anomalous event in response to a stimulus or confirmatory test. For example, a test engine 407 can include one or more processes for applying a stimulus and detecting a response or lack thereof from the body of the user. In another example, a test engine 407 can include one or more processes for prompting a monitored person or clinician to perform an activity, such as having the monitored person clench both fists so that a muscle response or lack thereof can be assessed.

The test engine 407 can be configured to initiate testing based on one or more inputs, such as a schedule, based on a user input, or a combination thereof. The schedule can be predetermined, automated, based on user preferences, periodic, non-periodic, or otherwise set by the system 1000 based on, for example, an estimated risk of stroke or medical anomaly. The test engine 407 can be configured to perform a plurality of different or the same tests, such as a thermoregulatory test and a muscle activity test on a plurality of schedules that may or may not be coordinated.

An output engine 409 can be configured to generate and/or transmit an output related to the determined risk of an anomalous medical event to a memory, display, speaker, computing device, or other communication or storage device. An output can include, but is not limited to an alert, a recommendation, a report, an assessment, the like or a combination thereof.

In some examples, the output engine 409 can be configured to generate an alert configured to capture attention of the monitored person and/or another person, such as an emergency medical service contact, a clinician, a person physically near the monitored person, a friend or family contact, or other person capable of providing help to the person in relation to a potential anomalous medical event. The alert can, for example, be audible, visual, haptic, or a combination thereof. In some examples, an alert can comprise multiple alerts and/or a continuous alert configured to continue to output until acknowledged. In some examples, an alert can be a single alert not configured to repeat and/or configured to turn off after a period of time. In some examples, an alert can be configured to output to multiple devices, contacts, and/or in multiple modes, such as audibly, visibly, haptically, or a combination thereof. In some examples, the output engine 409 can be configured to output a series of alerts, such as escalating alerts, such as if a risk is determined to increase, if a person has not been attended to, and/or if an alert has not been acknowledged. In some examples, the output engine 409 can be configured to generate different levels of alerts based on different levels of determined risk or confidence associated with the risk. For example, if a risk is determined to be of low confidence a different alert can be issued that if a risk is determined to be of high confidence.

In some examples, the output engine 409 can be configured to generate a report or assessment associated with the risk, such as a monitoring history of the monitored person, a history of features or identified features, a history of alerts, how long since the onset of an identified feature, feature, or alert, whether a person has been treated previously or actively for an anomalous medical event, the like or a combination thereof. The report or assessment can be transmitted, stored, or otherwise output to a cloud, locally to a monitoring or computing device, or a combination thereof. The report or assessment can be generated based on user request or input or periodically or continuously stored during monitoring of a person and/or in response to the generation of an alert or the identification of an anomalous medical event or a risk thereof.

In some examples, the output engine 409 can be configured to generate a recommendation for treatment based on, for example, an identified risk or other monitoring parameter of the patient. For example, if a patient is identified as having a rapid and/or significant increase in blood pressure, the output engine 409 can generate an alert and recommendation to administer treatment to the person.

### Test Algorithm

FIG. 5 illustrates an example test process 500 that can be implemented by a multivariate stroke detection system 1000 in order to generate an output, such as risk, alert, and/or treatment recommendation.

For example, process 500 of FIG. 5 relates to passive and/or active monitoring of a person for an anomalous medical event, such as a stroke. For example, a process 500 can be performed by a controller 400 in relation to continuous or spot-check monitoring or measurement of data of a person. In some examples, the process 500 can be performed continuously or semi-continuously, on a user requested basis, and/or on a scheduled basis, such as periodically over a set course of time and/or until an end condition is met, such as a sensor off event and/or a user input indicating a stop request.

In an example process 500, a controller 400 can receive data from one or more monitoring devices, sensors, and/or other sources in a multivariate stroke detection system 1000 at a step 501. For example, one or more monitoring devices or sensors can be configured to collect physiological data from a monitored person. The controller 400 can be in communication with the one or more devices or sensors to request and/or receive the collected physiological data. In addition or in the alternative, the controller 400 can be a part of the one or more devices and/or sensors.

The data monitoring devices or sensors can include, but are not limited to non-invasive physiological monitoring devices, such as at least one wearable device including sensors, such as PPG, EMG, EEG, ECG, EKG, DSA, GSR, temperature sensors, microphone sensors, cameras, the like or a combination thereof. Advantageously redundant sensors applied at bilateral locations of the body, such as a left lateral or right lateral side can be arranged to collect data from a person at a similar point in time so as to identify asymmetries in physiological data that can be relevant to identification of an anomalous medical event, such as a stroke. In some examples, sensors can be non-redundant and/or configured to measure systemic as opposed to bilateral physiological data.

The controller 400 can be configured to, at a step 501, administer a stimulus in order to generate test data for collection. The stimulus, can, for example, include one or more applied stimulus to the body of the patient, such as application of heat via one or more wearable devices coupled to a patient. The system 1000 can then collect data in relation to the stimulus, such as before, during, and after the stimulus is applied.

The controller 400 can be configured to, at a step 501, direct a clinician or user to perform a confirmatory test, such as a movement activity (e.g. clenching of both fists, raising of arms, etc.) via a user interface, which can include visual and/or audible instructions and/or an indicator reminding a clinician to administer such an exam. The system 1000 can be configured to collect data via, for example, one or more sensors coupled to the patient.

The controller 400 can, at step 501, additionally or alternatively be configured to access medical record data associated with a monitored person. The medical record information can be accessed based on stored data in an electronic medical record (EMR), selectively input by a user and/or clinician, and/or a combination thereof. The medical record data can, for example, include certain aspects of medical history, such as heart disease or other neurological conditions that can impact accuracy of a medical risk assessment. In some examples, the medical record data can include previous or presently calculated risk assessments, such as a previously calculated assessment by a system 1000 and/or a clinician or other user. In some examples, the medical record data can include an indication of whether the monitored person or patient is at risk for a stroke and/or is being monitored for neurological decline as a result of a recent stroke and/or if the patient is otherwise healthy. Other types of medical record information can also be accessed and/or utilized, such as patient demographic information, patient activity information, or other patient specific information.

At a step 503, the controller 400 can extract one or more features from the data collected at step 501. The one or more feature can include, but is not limited to the features identified herein, such as features related to heart rate variability (e.g., TINN or PNN50), skin temperature, sweat, muscle activity, and/or others. The extracted features can be systemic and/or bilateral. The extracted features may or may not be associated with the same temporal collection window. The extracted features may or may not be validated or otherwise processed for analysis. The controller 400 can be configured to pre-process the data from step 501 as part of or separately from a feature extraction step 503.

The controller 400 can extract and/or analyze features based at least in part on the data received at step 501. For example, the controller 400 can exclude analysis of features associated with muscle activity in a paralyzed patient based on medical record data and/or exclude analysis of features associated with heart rate variability in a patient with heart disease. The controller 400 can alternatively, in some examples, select different features for analysis. In some examples, a controller 400 can additionally or alternatively assign a lower confidence metric to a risk assessment based on features that can be impacted by non-stroke factors as a function of the patient specific medical record data.

At a step 505, the controller 400 can identify a risk and/or risk confidence based at least in part on the extract data feature(s) 503. The risk can include, but is not limited to a percentage risk of an anomalous medical event having occurred, will likely occur, or is presently occurring. In some cases, the risk can include a binary determination of whether or not a person recently experienced or is experiencing an anomalous medical event, such as a stroke. In some cases, the risk can include a classification of the medical event, such as type and/or location of stroke. A risk confidence can include, but is not limited to a weight and/or other metric associated with a level of accuracy of the risk assessment. The risk confidence can be impacted based on one or more factors that can impact the accuracy of the risk assessment, including but not limited to a strength of correlation of the feature with a medical event, a signal quality, patient specific information, the like or a combination thereof.

In some examples, the controller 400 can identify multiple risks and/or risk confidence values based on a plurality of extracted features. The risks can be determined based on a plurality of different pairings of features, different assessments, or a combination thereof. In some examples, the risks can be determined independently and/or combined to generate an overall risk assessment. The combination can be based at least in part on individual confidence and/or weighting metrics.

In some examples, the controller 400 can identify a risk, at least in part, based on a mapping of extracted features to a predetermined mapping of features determined based on experimental data. In some examples, the controller 400 can identify a risk, at least in part, based on a tracking and/or change in trends or values of an individual feature and/or set of features tracked in relation to an individual patient in addition to or in the alternative to a mapping associated with a plurality of different individuals.

In some examples, the risk can be associated with a noted decline in neurological condition in addition to or in the alternative to a stroke event. The risk can be based at least in part on a tracking of at least one feature of an individual.

At a step 507, the controller 400 can determine whether a confidence metric associated with the out of step 505 is of sufficient quality to produce an output. If the confidence metric falls below a threshold, the controller 400 can prompt the system 1000 for more data (e.g., return to step 501) and/or determine a different risk assessment based at least in part on a different feature or set of features (e.g., return to step 503). In some examples, the controller 400 can identify a risk based at least in part on a series of risk assessments. For example, a controller 400 can identify a first risk based on a first individual feature and/or set of features. If that feature and/or set of features does not exceed a threshold confidence, the controller 400 can identify a second risk based on a second feature and/or a second set of features.

At a step 509, the controller 400 can determine whether a risk passes a threshold. If the risk passes the threshold, the controller can output an alert at step 511 and/or optionally generate a treatment recommendation at a step 513. If the risk does not pass the threshold, then the controller can perform one or more of the previous steps of the process. For example, the controller can collect more data, extract new and/or different features, calculate a new or different risk, or a combination thereof. The threshold can include, in some examples, a binary check of whether the risk is indicative of a stroke event or not. The threshold can include, in some examples, a value of risk associated with a greater likelihood of stroke, such as greater than approximately 50 percent chance, 60 percent chance, 70 percent chance or greater or less than that amount. The threshold at step 509 can be set or changed based on any number of factors, such as a user input, a predetermined value, a confidence measure associated with the feature extraction and/or risk determination, the like or a combination thereof. The threshold can include a series of thresholds that trigger a series of escalating alerts, such as a low level alert for a 40 percent chance of stroke, a mid level alert for a 60 percent chance of stroke, and a high level alert for a greater than 80 percent chance of stroke. However, other thresholds and/or alert correlations are also possible.

The outputs of the process, such as risk(s), confidence metric(s), recommendation(s), and alert(s) can be communicated to one or more devices so as to call attention to patient and/or a caregiver or other person in order to increase the likelihood of the patient receiving appropriate medical attention. For example, an alert can be audible to facilitate persons in the vicinity of the patient to understand that help can be needed. In another example, an alert can be pushed to a clinician device or patient monitor to facilitate notifying a treating physician that they should initiate treatment. One or more of the output(s) can additionally or alternatively be stored in a memory of a device and/or the cloud, such as in an electronic medical record, in local memory of a monitoring device and/or mobile computing device, the like or a combination thereof.

### Example Thee-Dimensional Feature Mapping

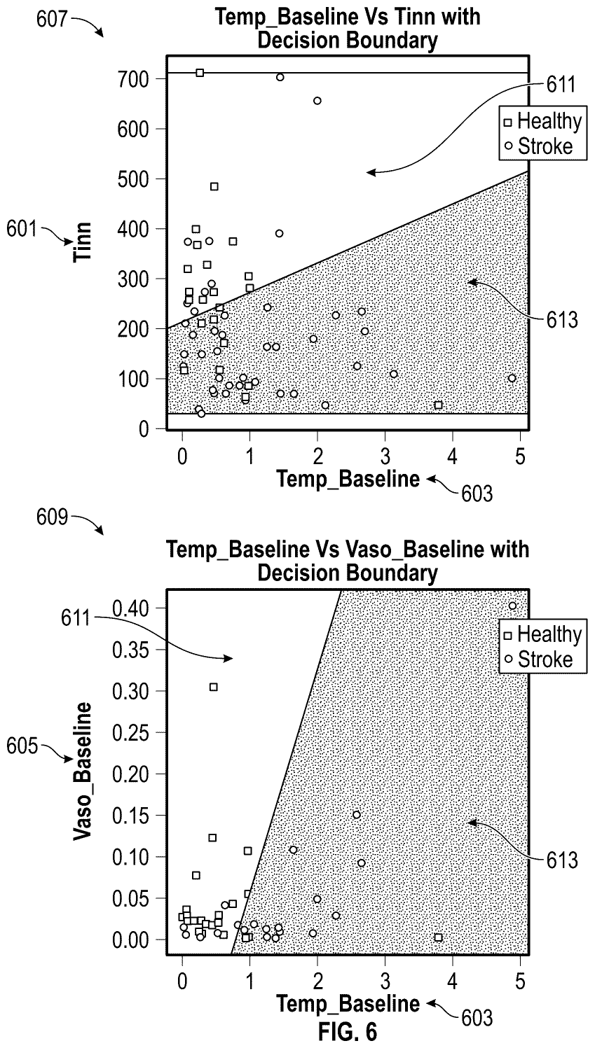

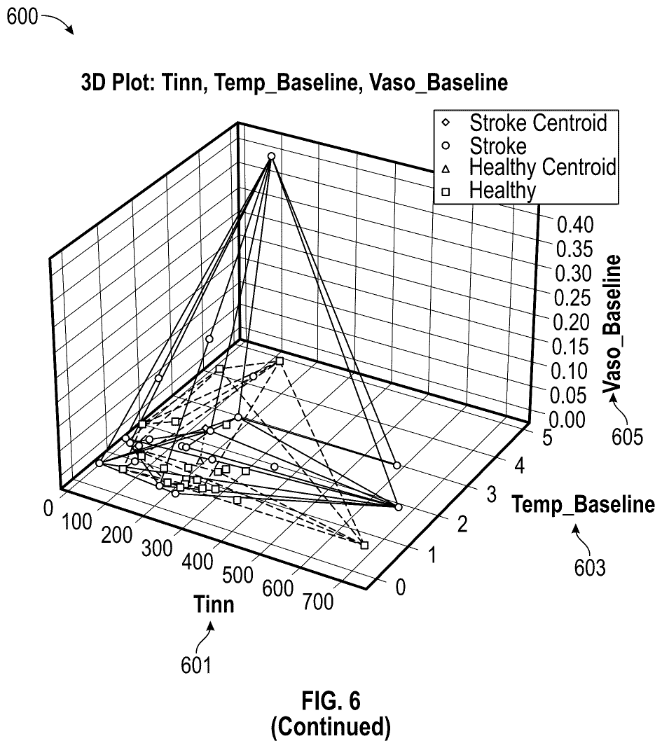

FIGS. 6-12 illustrate additional example feature mappings that can be utilized as part of a risk classification by a system 1000 as a basis of comparison for data of monitored persons. As mentioned above, the system 1000 can compare data from a monitored person or patient to the illustrated mappings in to help identify and/or classify a potential event experienced by the monitored person or patient. The feature mapping examples disclosed herein can relate to intra-individual feature mapping such that each data point on the mappings relate to a different individual either having had a stroke event or a non-stroke event, having prior medical history or no prior medical history, etc.

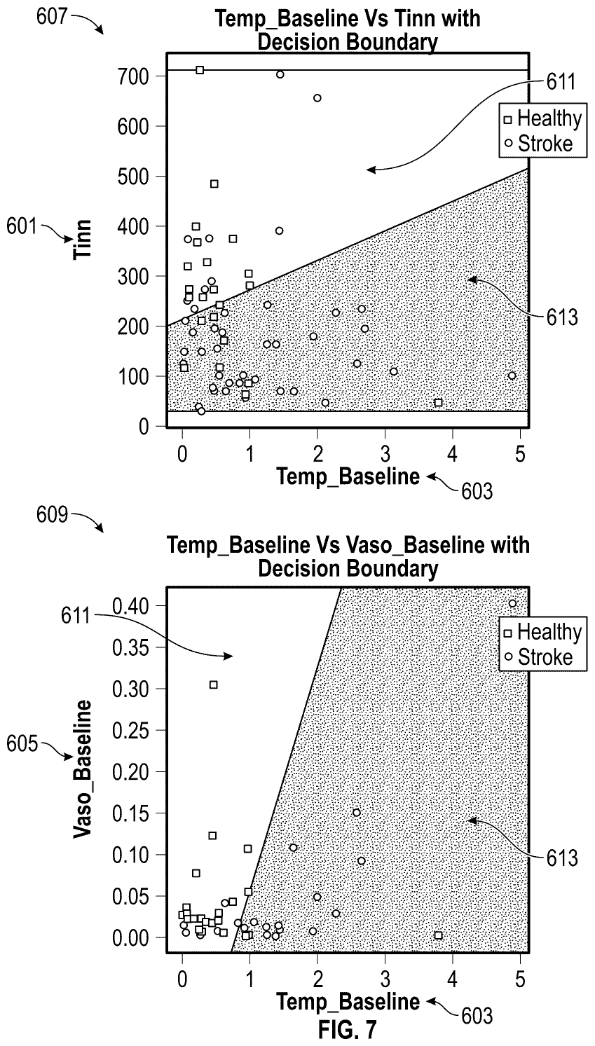

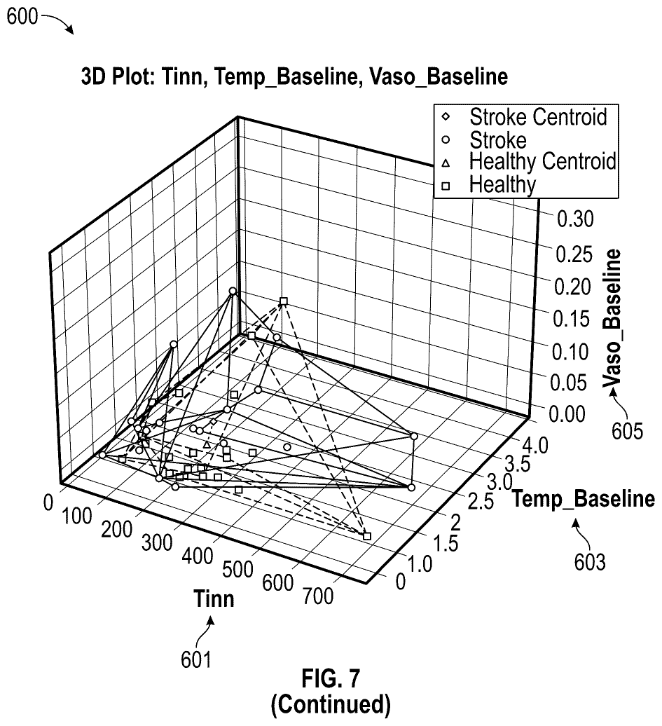

FIG. 6 illustrates an example feature mapping 600 of data values for comparing triangular interpolation of N-N intervals in an ECG signal 601, a baseline skin temperature 603, and a baseline vasodilation 605, and FIG. 7 illustrates the feature mapping 600 of FIG. 6 with outliers removed. The feature mapping 600 can be illustrated as a 3D scattered dot plot, which provides a visual representation between triangular interpolation of the N-N intervals (TINN) from ECG signals (X-axis), the baseline skin temperature (Y-axis), and the baseline vasodilation (Z-axis) while distinguishing between stroke patients and healthy individuals. The feature mapping 600 can be used to create a classifier for identifying healthy individuals and individuals who are at risk of having a stroke. The classifier can use decision boundaries derived from the feature mapping 600 to categorize new patient data points based on their proximity to known risk groupings.

The feature mapping 600 can be illustrated by combining from two separate 2D scatter plots 607, 609, each analyzing different pairwise relationships among TINN, baseline temperature, and baseline vasodilation. The first 2D scatter plot 607 can compares baseline skin temperature 603 (X-axis) and TINN 601 (Y-axis), showing how heart rate variability (HRV) varies with changes in body temperature. As illustrated, the shaded area 611 of the scatter plot 607 can associated with a grouping of patients being classified as having healthy physiological parameters (e.g., those having higher TINN values and lower skin temperature values) in comparison to the area 613 of the plot associated with patients classified as having stroke related physiological parameters (e.g., those having lower TINN values and higher skin temperature values). A boundary decision line can be drawn between the shaded area 611 and area 613 indicating a decision threshold. Thus, those patients having a classifier of high TINN values and lower skin temperatures values can be grouped in into shaded area 611 and those having the inverse classifier can be grouped into area 613. The second 2D scatter plot 609 examines baseline skin temperature 603 (X-axis) and baseline vasodilation 605 (Y-axis), illustrating how vascular responses are influenced by thermoregulation. Similar to the scatter plot 607, the scatter plot 609 can include the shaded area 611 associated with a grouping of patients being classified as having healthy physiological parameters (e.g., those having higher baseline vasodilation values and lower skin temperature values) in comparison to the area 613 of the plot associated with patients classified as having stroke related physiological parameters (e.g., those having lower baseline vasodilation values and higher skin temperature values). A boundary decision line can also be drawn between the shaded area 611 and area 613 indicating a decision threshold. Therefore, those patients having a classifier of higher baseline vasodilation values and lower skin temperature values can be grouped in into shaded area 611 and those having the inverse classifier can be grouped into area 613. By integrating these two perspectives into a 3D visualization, the combined plot provides a more comprehensive view of how these three physiological parameters interact, particularly in distinguishing between stroke and healthy individuals.

As shown in FIGS. 6 and 7, the X-axis represents TINN values, ranging from 0 to 700, which quantify heart rate variability (HRV). TINN is derived from the ECG signal and represents the width of the distribution of N-N intervals fitted to a triangular model. Variations in N-N intervals, derived from ECG signals using triangular interpolation, which smooths and estimates heart rate variability (HRV) by fitting a triangular function over the distribution of N-N intervals. This can help quantify autonomic nervous system activity and assess cardiovascular function. Higher TINN values (e.g., closer to 700 as shown in FIG. 6) indicate greater HRV, which can be associated with better autonomic regulation and cardiovascular adaptability, while lower values suggest reduced HRV, often linked to autonomic dysfunction, particularly in stroke patients. The Y-axis represents baseline temperature (shown ranging from 0 to 5) as a normalized or scaled version of body temperature, indicating difference in thermoregulation between stroke and healthy individuals. The Z-axis represents baseline vasodilation (shown ranging from 0 to 0.40) as a fractional change or percentage. For example, a value of 0.40 can indicate a 40% increase in vessel diameter or blood flow from baseline, reflecting vascular adaptability and autonomic control.

In feature mapping 600, individual data points can be categorized into stroke and healthy groups. Centroids for both groups can be calculated and marked, representing the central tendency of stroke and healthy individuals. This can illustrate physiological differences, especially if stroke patients exhibit lower HRV (lower TINN values), altered thermoregulation (temperature differences), or impaired vasodilation. By analyzing the spatial distribution of the data points, trends can be identified in HRV (X-axis), thermoregulation (Y-axis), and vascular response (Z-axis). A separation between the stroke and healthy centroids can suggest a physiological differences, while overlapping clusters might indicate more subtle variations.

The selection of data for skin temperature can be a window of data over a period of time, such as 10, 20, 30, 40, 50, or 60 seconds or more. The period of time can relate to an amount of time during which the data is of sufficient quality to generate a reasonable result. For example, the data can be processed, filtered and/or windowed to generate a Signal to Noise Ratio (SNR) satisfying a threshold quality to map. The period of time can be some combination of metrics, such as a period of time during which the stimulus is applied and during which the SNR is greater than a threshold. Other periods or windows can additionally or alternatively be utilized based on other factors impacting the collection and/or analysis of data.

Mapped feature types may or may not be collected within the same or even overlapping windows of data collection. In some examples, mapped feature types can be collected in the same time period. In some examples, mapped feature types can be collected in different time periods. For example, a heart rate variability metric, such as TINN can be collected up to 30 minutes or greater after a skin temperature metric or a vasodilatory metric is collected. However, in some cases, feature types can be correlated in time to include the same or overlapping windows of data collection. Whether mapped feature types are collected in the same window may or may not be based on likelihood of physiological changes within that window. For example, a heart rate variability metric can be less likely to change within a 30 minute period than a muscle activity metric and thus, the data collection windows may not need to overlap.

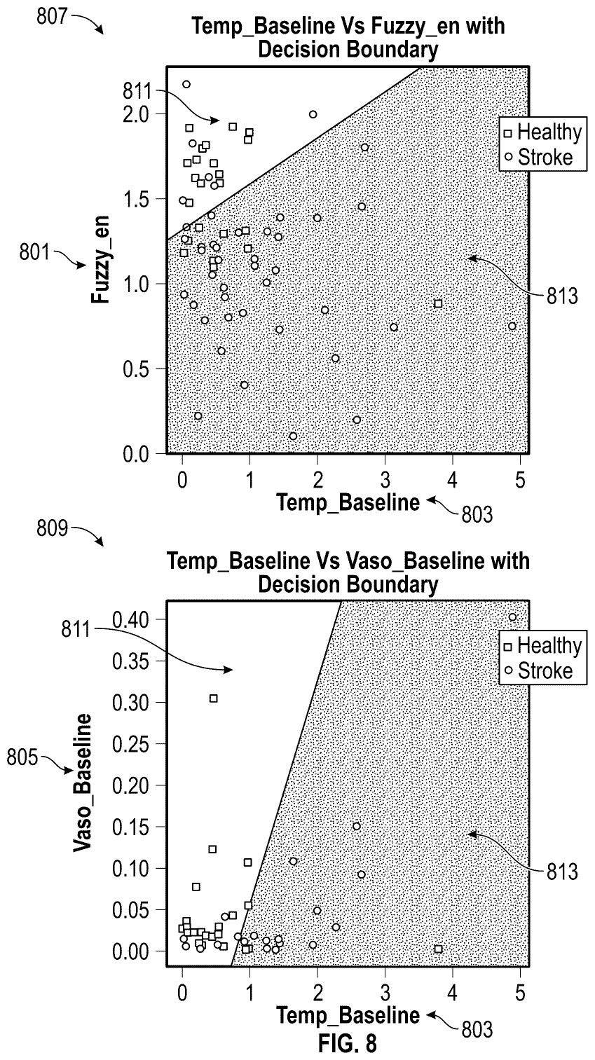

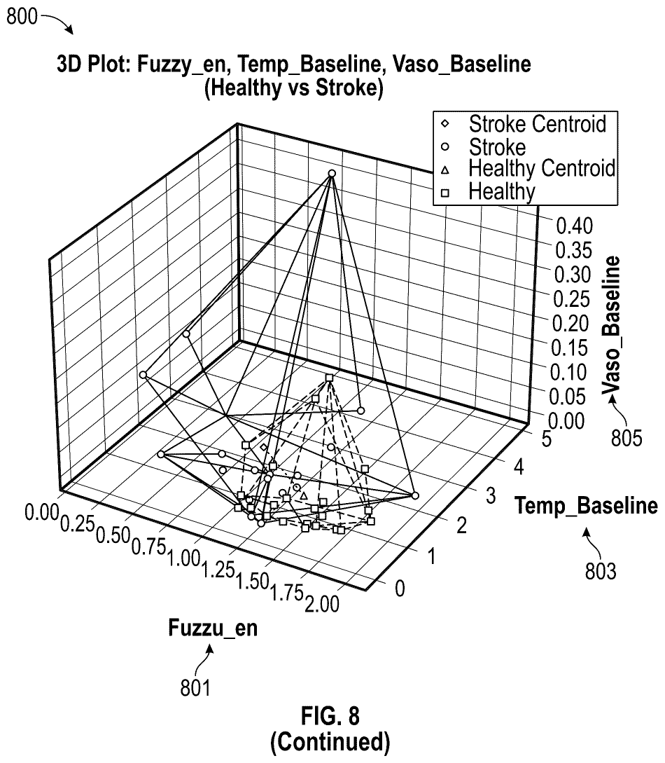

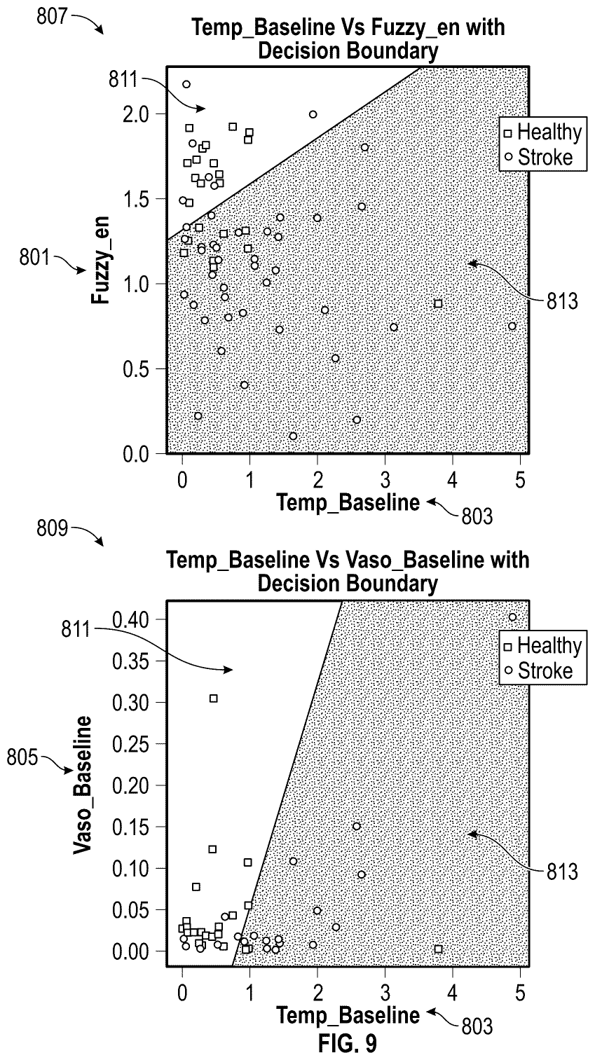

FIG. 8 illustrates another example feature mapping 800 of data values for comparing fuzzy entropy 801, a baseline skin temperature 803, and a baseline vasodilation 805, and FIG. 9 illustrates the feature mapping 800 of FIG. 8 with outliers removed. Rather than comparing triangular interpolation of N-N intervals in an ECG signal versus baseline skin temperature and baseline vasodilation as shown in FIG. 6, fuzzy entropy can be compared in relation to baseline skin temperature and baseline vasodilation in FIG. 8. FIG. 9 illustrates the feature mapping 800 of FIG. 8 with outliers removed. The feature mapping 800 can be illustrated as a 3D scattered dot plot, which provides a visual representation of the relationships between fuzzy entropy 801 (X-axis), a baseline skin temperature 803 (Y-axis), and baseline vasodilation 805 (Z-axis), distinguishing between stroke patients and healthy individuals. Similar to the feature mapping 600, the feature mapping 800 can also be used to create a classifier for identifying healthy individuals and individuals who are at risk of having a stroke.

FIG. 8 can be illustrated by combining two separate 2D scatter plots 807, 809, each analyzing different pairwise relationships among fuzzy entropy 801, baseline skin temperature 803, and baseline vasodilation 805. The first 2D scatter plot 807 can compares baseline skin temperature 803 (X-axis) and fuzzy entropy 801 (Y-axis), showing how heart rate variability (HRV) changes in response to variations in body temperature. As illustrated, the shaded area 811 of the scatter plot 807 can associated with a grouping of patients being classified as having healthy physiological parameters (e.g., those having higher fuzzy entropy values and lower skin temperature values) in comparison to the area 813 of the plot associated with patients being classified as having stroke related physiological parameters (e.g., those having lower fuzzy entropy values and higher skin temperature values). A boundary decision line can be drawn between the shaded area 811 and area 813 indicating a decision threshold. Thus, those patients having a classifier of high fuzzy entropy values and lower skin temperatures values can be grouped in into shaded area 811 and those having the inverse classifier can be grouped into area 813. The second 2D scatter plot 809 examines baseline skin temperature 803 (X-axis) and baseline vasodilation 805 (Y-axis), demonstrating how changes in body temperature impact vascular responses and the body's ability to regulate blood flow. Similar to the scatter plot 807, the scatter plot 809 can include the shaded area 811 associated with a grouping of patients being classified as having healthy physiological parameters (e.g., those having higher baseline vasodilation values and lower skin temperature values) in comparison to the area 813 of the plot associated with patients classified as having stroke related physiological parameters (e.g., those having lower baseline vasodilation values and higher skin temperature values). A boundary decision line can also be drawn between the shaded area 811 and area 813 indicating a decision threshold. Therefore, those patients having a classifier of higher baseline vasodilation values and lower skin temperature values can be grouped in into shaded area 811 and those having the inverse classifier can be grouped into area 813. By integrating these two perspectives into a 3D visualization, the combined plot provides a more comprehensive view of how these three physiological parameters interact, particularly in distinguishing between stroke and healthy individuals.

As shown in FIGS. 8 and 9, the X-axis can represent fuzzy entropy values (ranging from 0 to 2.00), which quantify heart rate variability (HRV). Fuzzy entropy is a measure of the complexity and irregularity of the heartbeat intervals, derived from the ECG signal. Higher fuzzy entropy values (e.g., closer to 2.00 as shown in FIG. 6) indicate greater HRV, which can be typically associated with better autonomic regulation and cardiovascular adaptability, while lower values suggest reduced HRV, often linked to autonomic dysfunction, particularly in stroke patients. The Y-axis represents baseline temperature (shown ranging from 0 to 5) as a normalized or scaled version of body temperature, indicating difference in thermoregulation between stroke and healthy individuals. The Z-axis represents baseline vasodilation (shown ranging from 0 to 0.40) as a fractional change or percentage. For example, a value of 0.40 can indicate a 40% increase in vessel diameter or blood flow from baseline, reflecting vascular adaptability and autonomic control.

In feature mapping 800, individual data points can be categorized into stroke and healthy groups. Additionally, centroids for both groups can be calculated and marked, representing the central tendency of stroke and healthy individuals. This can illustrate physiological differences, particularly if stroke patients exhibit lower fuzzy entropy (lower HRV values), altered thermoregulation (temperature differences), or impaired vasodilation. By analyzing the spatial distribution of the data points, trends can be identified in HRV (X-axis), thermoregulation (Y-axis), and vascular response (Z-axis). A separation between the stroke and healthy centroids can suggest a physiological differences, while overlapping clusters might indicate more subtle variations.

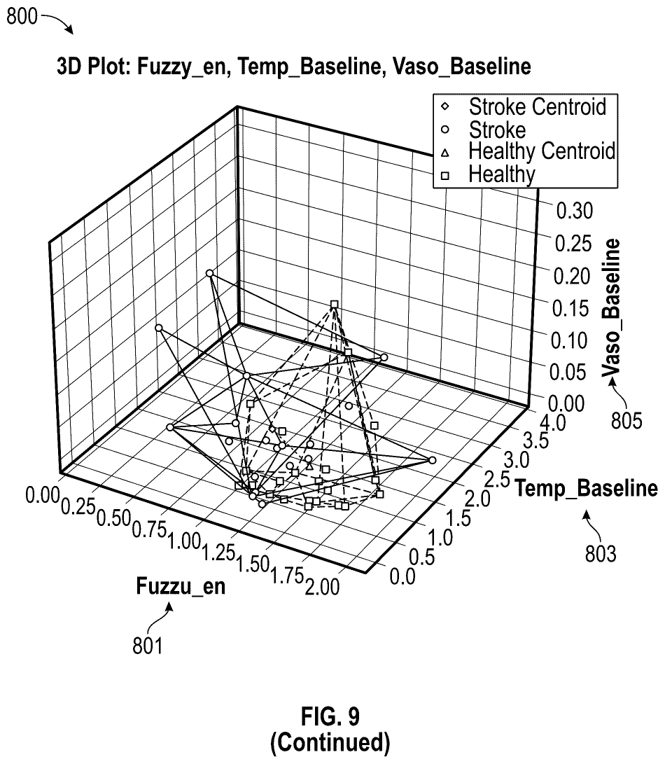

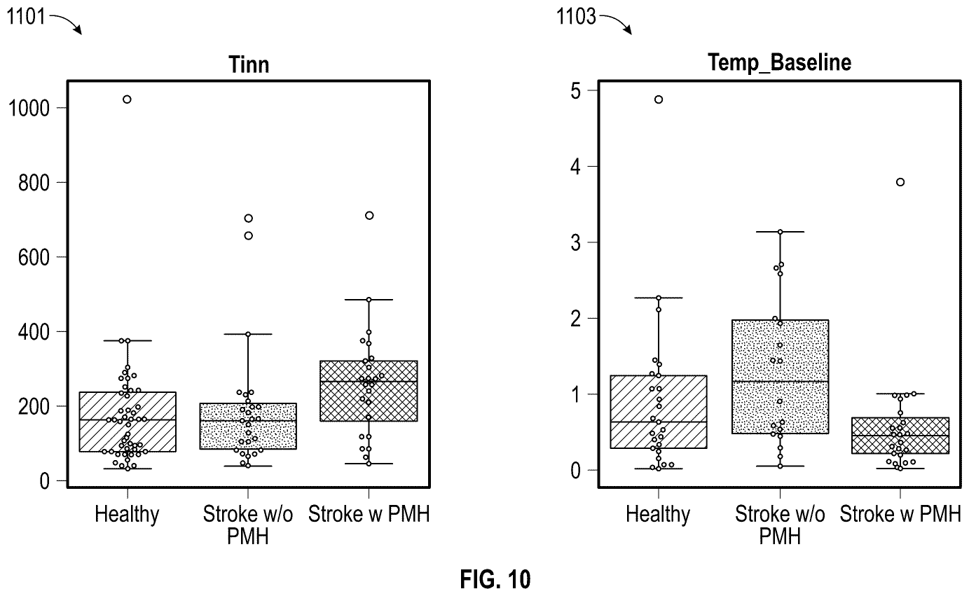

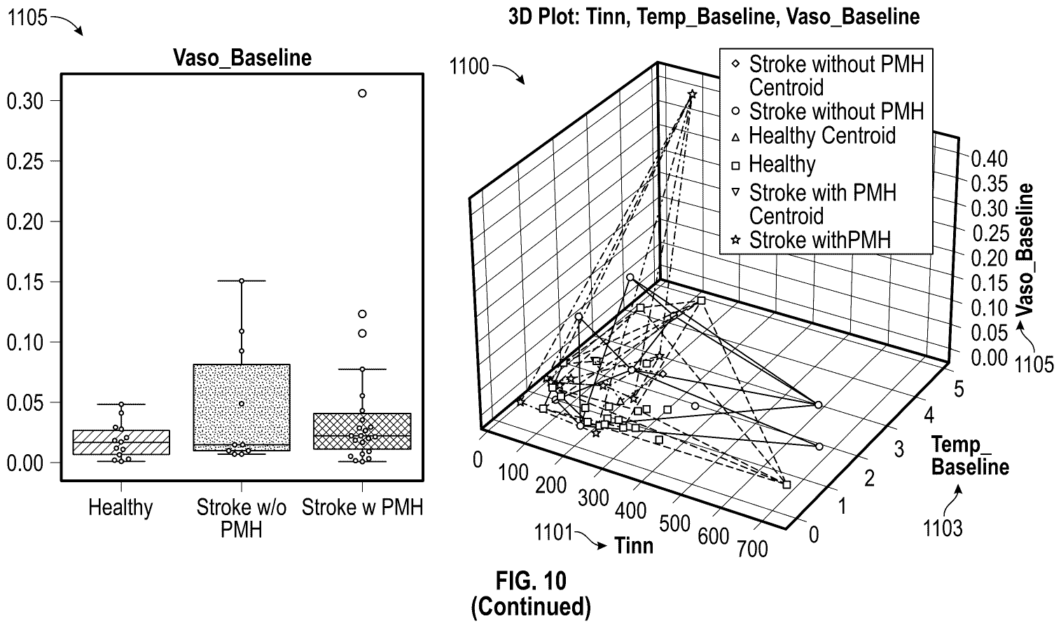

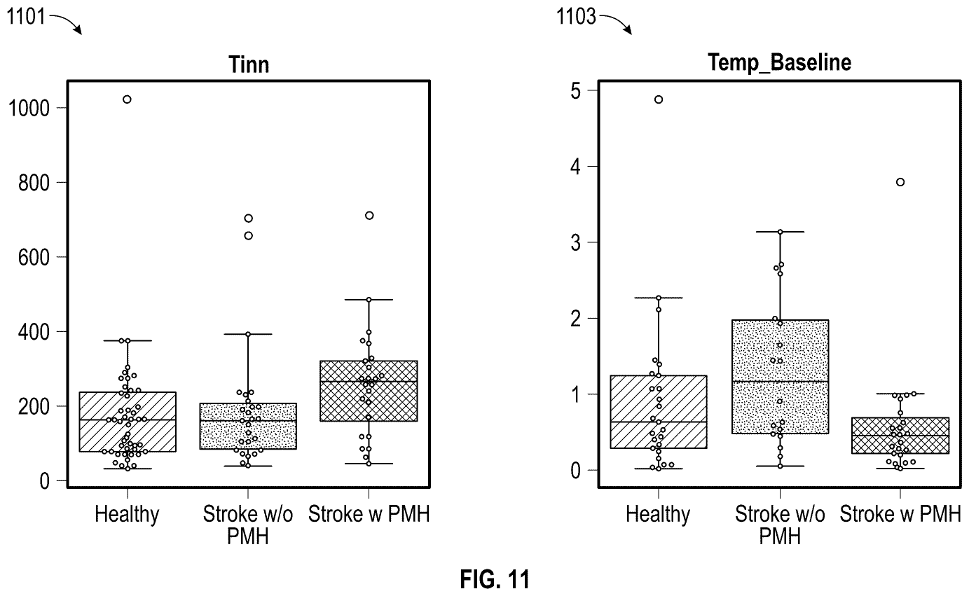

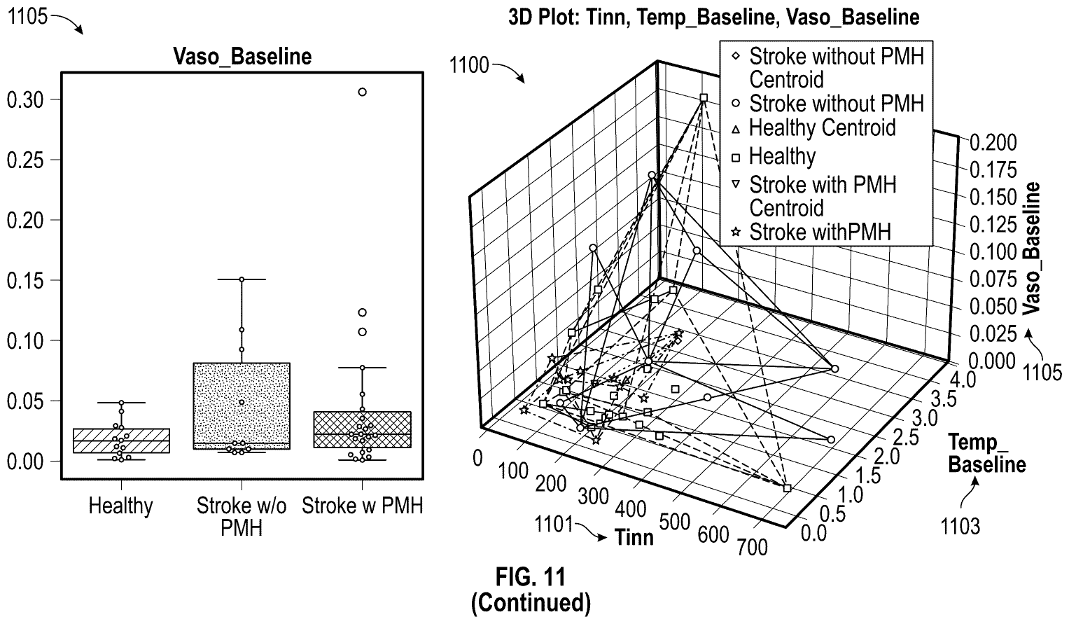

FIG. 10 illustrates an example feature mapping 1100 of data values for comparing triangular interpolation of N-N intervals in an ECG signal 1101, a baseline skin temperature 1103, and a baseline vasodilation 1105, and FIG. 11 illustrates the feature mapping 1000 of FIG. 10 with outliers removed. The feature mapping 1100 can be similar and/or identical to the feature mapping 600 except that the feature mapping 1100 also considers prior medical history in analyzing the ECG signal 1101, baseline skin temperature 1103, and/or baseline vasodilation 1105. In considering prior medical history in view of the feature mapping, it can help determine whether prior medical history impacts stroke physiology and whether it can serve as a classifier for stroke determination. Each of the ECG signal 1101, baseline skin temperature 1103, and/or baseline vasodilation 1105 can be further distinguished by whether the data points are identified as healthy, stroke without prior medical history, and/or stroke with prior medical history, as shown by the boxplots in FIGS. 10 and 11.

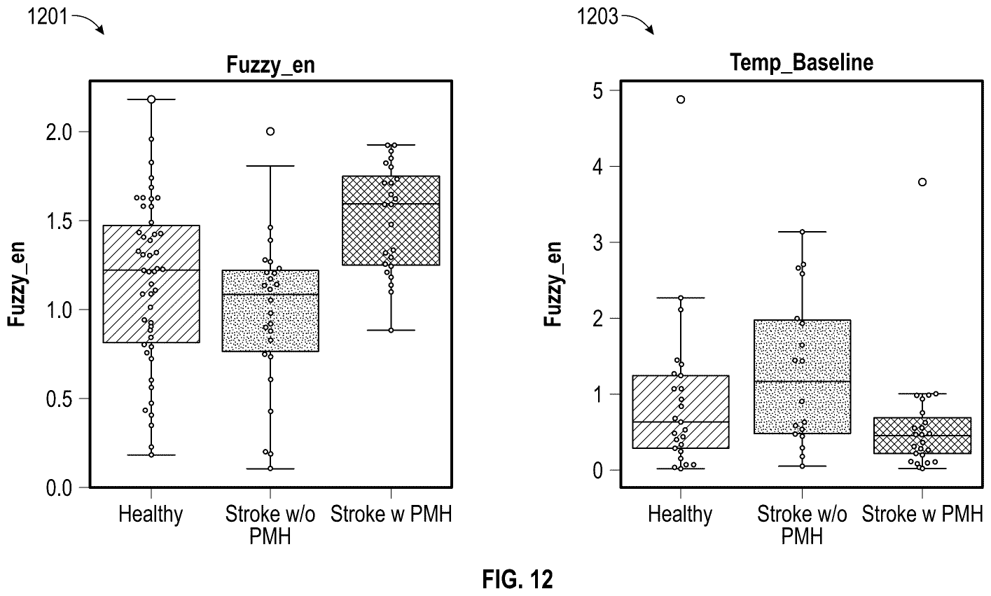

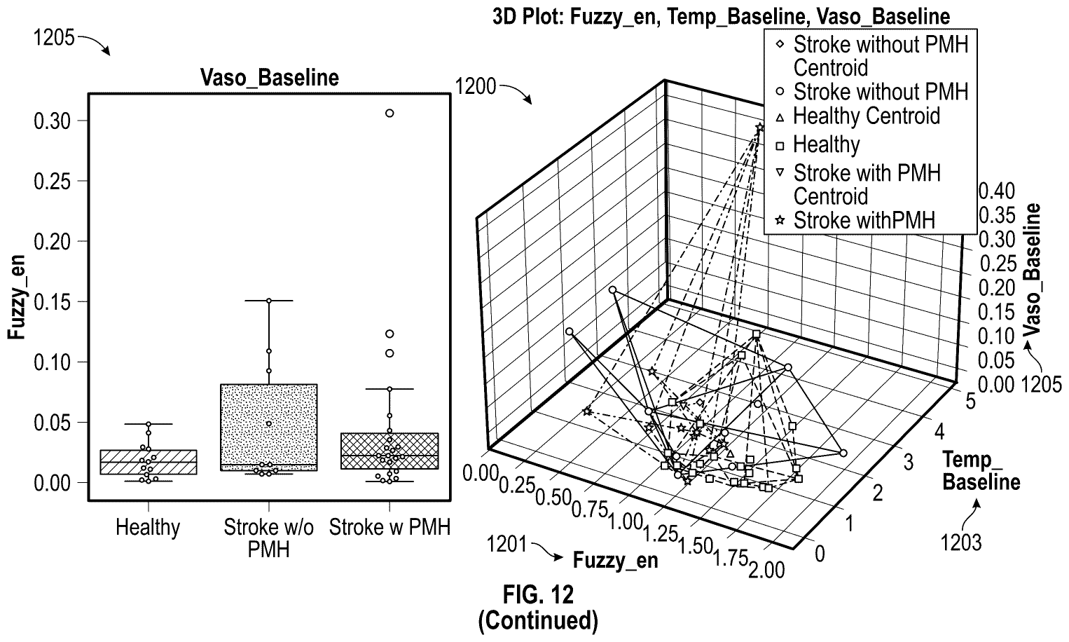

FIG. 12 illustrates another example feature mapping 1200 of data values for comparing fuzzy entropy 1201, a baseline skin temperature 1203, and a baseline vasodilation 1205. The feature mapping 1200 can be similar and/or identical to the feature mapping 800 except that the feature mapping 1200 also considers prior medical history in analyzing the fuzzy entropy 1201, baseline skin temperature 1203, and/or baseline vasodilation 1205. In considering prior medical history, it can help determine whether having prior medical history has an impact on stroke physiology. Each of the fuzzy entropy 1201, baseline skin temperature 1203, and/or baseline vasodilation 1205 can be further distinguished by whether the data points are identified as healthy, stroke without prior medical history, and/or stroke with prior medical history, as shown by the boxplots in FIG. 12.

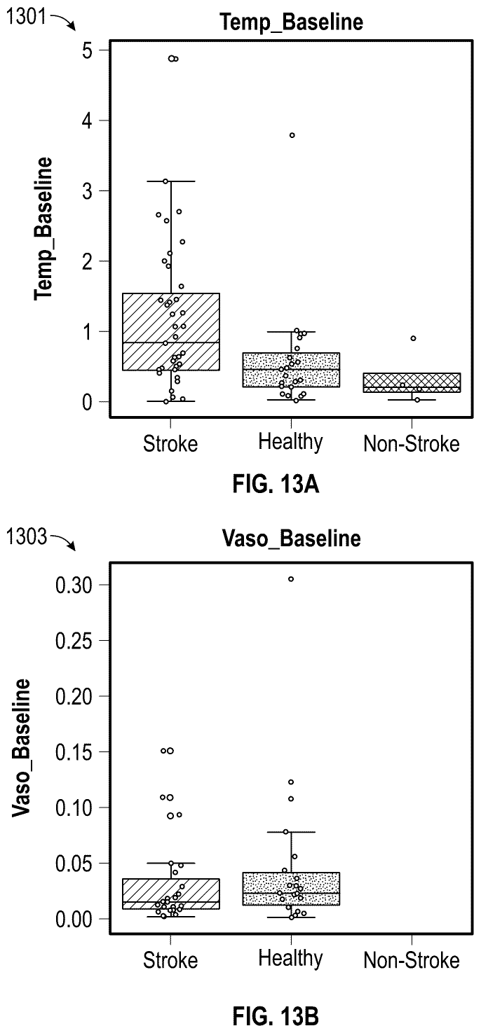

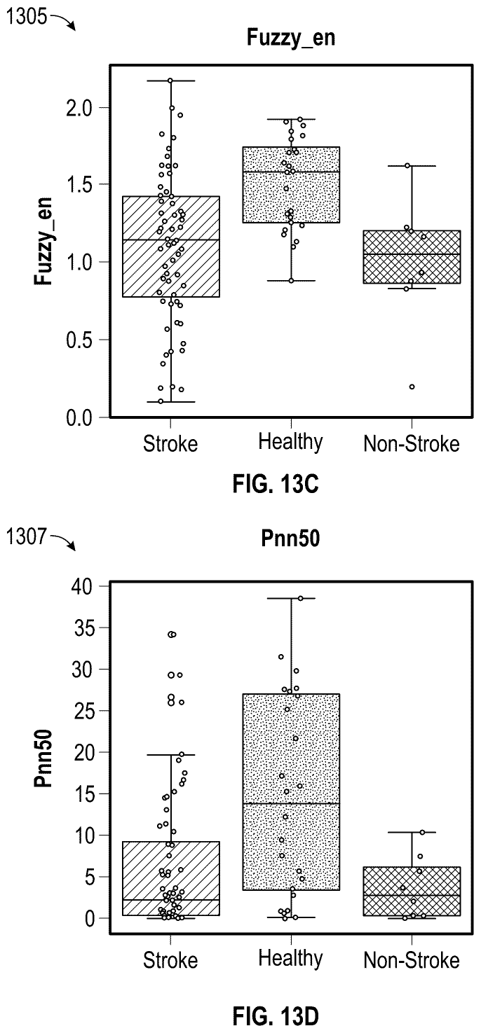

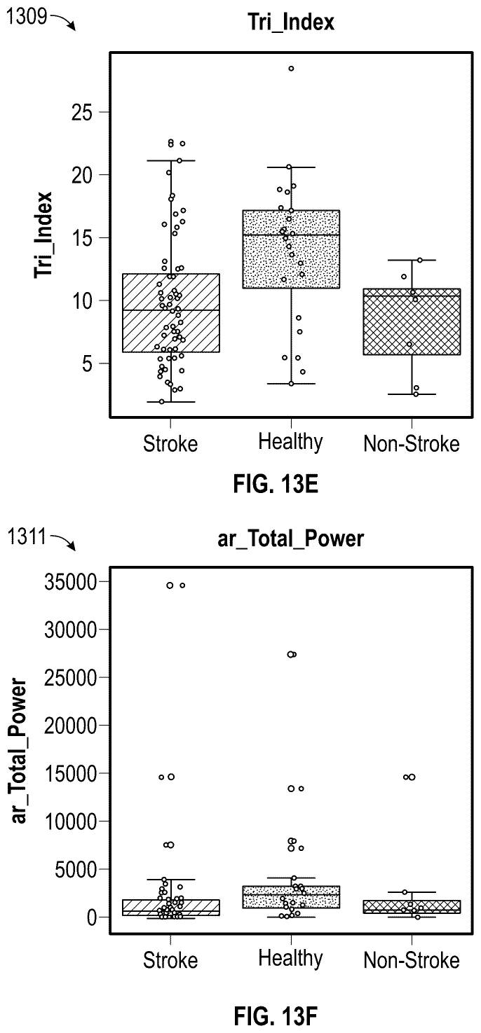

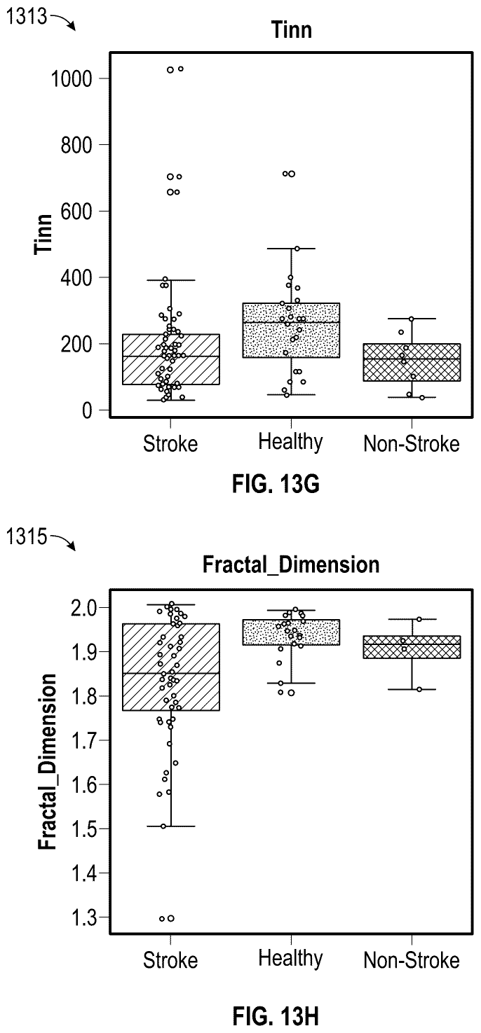

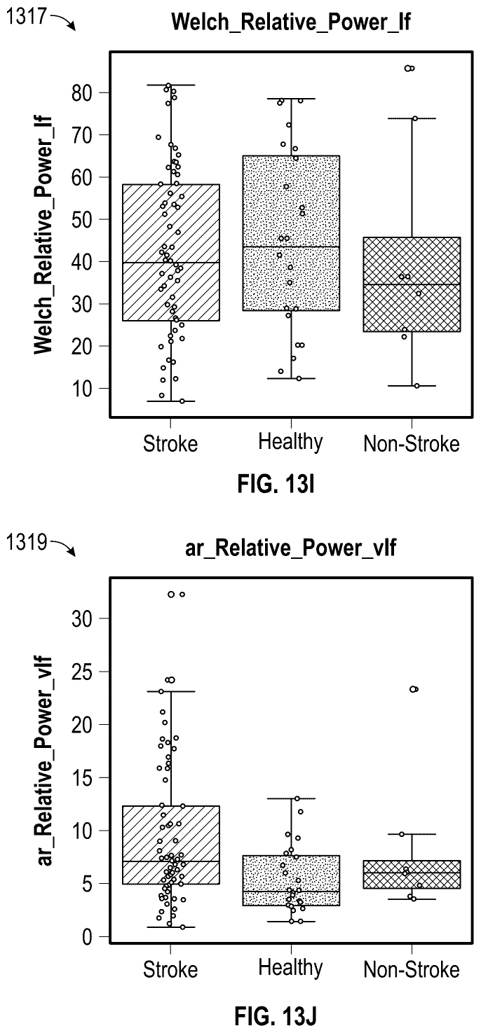

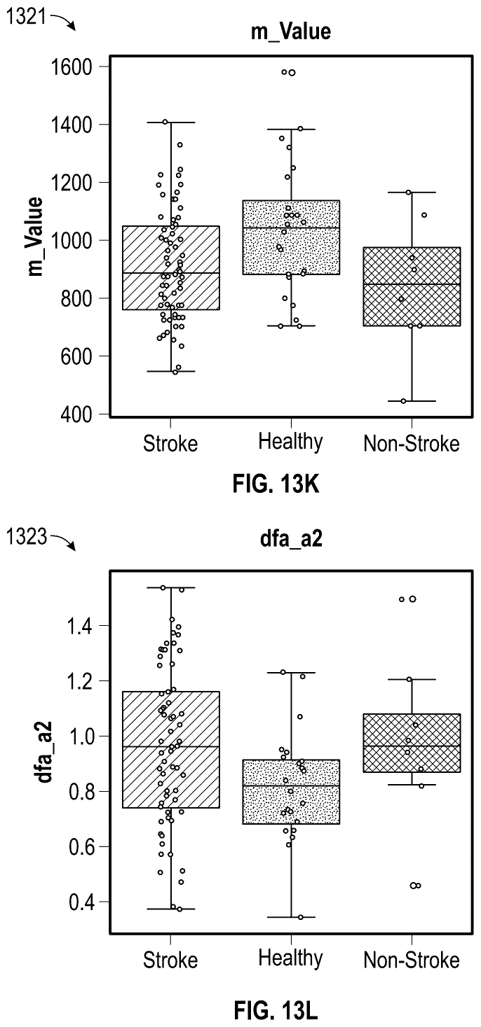

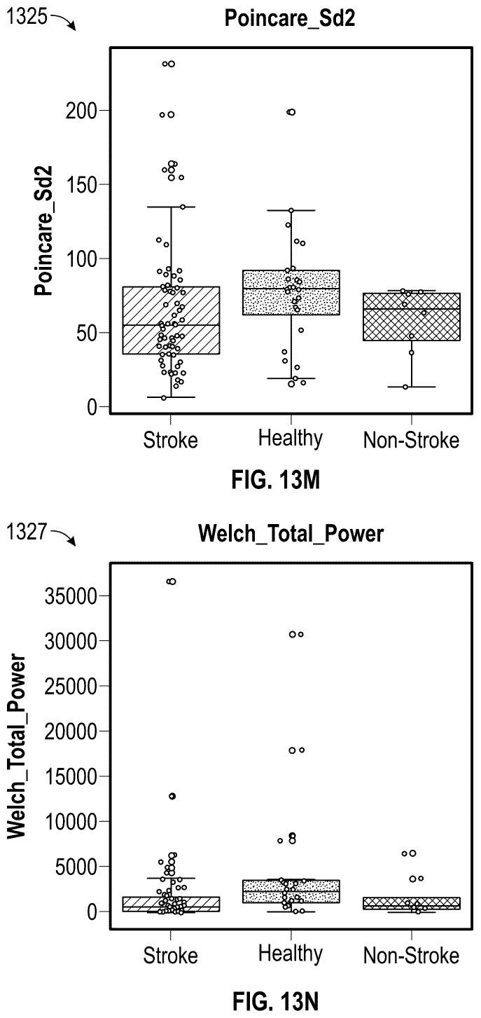

FIGS. 13A-13N illustrates various boxplots of physiological parameters considered in descriptions of FIGS. 6 to 12, which utilized to create classifications for patients. These boxplots can highlight differences between patient groups, helping to identify features that contribute to classification. By examining parameters such as heart rate variability, blood pressure fluctuations, oxygen saturation levels, and thermoregulation asymmetry, the classifications for patients can be refined to enhance diagnostic accuracy.

As shown in FIG. 13A, a boxplot 1301 can illustrate the distribution of baseline temperature values across stroke patients, individuals identified as healthy, and non-stroke patients with other conditions. Baseline temperature, the body's core temperature at rest, serves as a biomarker for physiological abnormalities. Each plot summarizes statistical measures, including the median, interquartile range (IQR), variability, and outliers. Whiskers extend from the box to show variability beyond the IQR, while outliers represent extreme values. Comparing the distributions of boxplot 1301 can help distinguish stroke patients from others, as stroke events can affect thermoregulatory responses, making temperature-based assessments useful for detection and classification.

The boxplot 1303 of FIG. 13B illustrates the distribution of vasodilation baseline values across stroke patients and individuals identified as healthy. Vasodilation baseline reflects the body's resting state of blood vessel dilation and serves as a biomarker for physiological abnormalities. The boxplot 1303 illustrates the range and variation of vasodilation baseline values within each group, allowing for a comparison between stroke patients and healthy individuals. By examining the distribution of these values, boxplot 1303 illustrates patterns that can be indicative of disruptions in vascular function. Since changes in vasodilation baseline are linked to impaired blood vessel regulation, this analysis can help identify differences in vascular responses between stroke patients and healthy individuals, supporting the potential use of vasodilation baseline as a diagnostic tool for stroke detection.

In FIG. 13C, the boxplot 1305 illustrates the distribution of fuzzy entropy values across stroke patients, individuals identified as healthy, and non-stroke patients with other conditions. Fuzzy entropy quantifies the complexity or irregularity of physiological signals, serving as a biomarker for physiological abnormalities. It ranges between 0.0 and 2.0, where lower values indicate more regular and predictable signals, and higher values reflect greater complexity and variability in the physiological response. In the context of stroke, changes in fuzzy entropy can signal disruptions in the body's normal regulatory processes. For example, stroke can cause irregularities in brain activity or cardiovascular function, leading to increased complexity in physiological signals. The boxplot 1305 can identify patterns that differentiate stroke patients from healthy individuals or non-stroke patients.

The boxplot 1307 illustrated in FIG. 13D shows the distribution of PNN50 (Percentage N-N intervals >50 msec) values across stroke patients and individuals identified as healthy. PNN50 reflects the percentage of consecutive heartbeats where the interval between them exceeds 50 milliseconds and serves as a biomarker for physiological abnormalities related to heart rate variability. The boxplot 1307 shows the range and variation of PNN50 values within each group, facilitating a direct comparison between stroke patients and healthy individuals. Differences in the distribution of PNN50 values can reveal disturbances in autonomic regulation, which can be impacted by stroke. Alterations in PNN50 levels could provide valuable insight into cardiovascular dysfunction associated with stroke, offering potential for its use in early detection and classification.

As shown in FIG. 13E, the boxplot 1309 illustrates the distribution of triangular index values across stroke patients and individuals identified as healthy. The triangular index is a time-domain measure of heart rate variability that represents the total number of N-N intervals divided by the height of the histogram of all N-N intervals, serving as a biomarker for autonomic nervous system function. By visualizing the spread and concentration of triangular index values, the boxplot 1309 illustrates how autonomic modulation of heart rate differs between stroke patients and healthy individuals. Deviations in the triangular index can reflect impairments in parasympathetic and sympathetic balance, which are observed in stroke-related dysautonomia.

In FIG. 13F, the boxplot 1311 illustrates the distribution of total power LF/HF ratio values across stroke patients and individuals identified as healthy. The total power LF/HF ratio is a frequency-domain measure of heart rate variability that quantifies the balance between low-frequency (LF) and high-frequency (HF) components, representing autonomic nervous system activity. The boxplot 1311 provides differences in autonomic regulation between stroke patients and healthy individuals by visualizing the spread and concentration of total power LF/HF ratio values. Shifts in the LF/HF ratio can indicate altered sympathetic and parasympathetic control, reflecting autonomic dysfunction associated with stroke.

The boxplot 1313 of FIG. 13G represents the distribution of TINN (Triangular Interpolation of N-N Intervals) values across stroke patients and healthy individuals (see FIGS. 10 and 11). TINN is a time-domain parameter of heart rate variability that estimates the width of the distribution of N-N intervals, offering a measure of autonomic function. The boxplot 1313 illustrates variations in TINN values, allowing for the comparison of autonomic control between stroke patients and healthy individuals. Changes in TINN can suggest altered cardiac autonomic regulation, which can be linked to disruptions in parasympathetic and sympathetic activity following a stroke.

As shown in FIG. 13H, the boxplot 1315 illustrates the distribution of fractal dimension values across stroke patients and healthy individuals. Fractal dimension is a nonlinear measure of heart rate variability that quantifies the complexity of heartbeat fluctuations, reflecting the regulatory mechanisms of the autonomic nervous system. It represents the degree of self-similarity and adaptability in cardiovascular dynamics, where higher values suggest a more responsive system, and lower values indicate a loss of complexity associated with autonomic dysfunction. The boxplot 1317 can illustrate differences in fractal dimension values between stroke patients and healthy individuals, allowing for an assessment of how stroke can impact heart rate dynamics. Analyzing these variations can help characterize changes in autonomic function, which could be linked to disruptions in physiological control mechanisms following a stroke.

In FIG. 13I, the boxplot 1317 illustrates the distribution of Welch relative power LF values across stroke patients and healthy individuals. Welch relative power LF is a frequency-domain metric that assesses the low-frequency component of heart rate variability, providing an indication of the balance between sympathetic and parasympathetic nervous system activity. Elevated values of the LF component can suggest stronger sympathetic influence, while lower values are associated with a reduced autonomic response. The boxplot 1317 can compare the distribution of these values between stroke patients and healthy individuals, showing the potential shifts in autonomic regulation due to stroke. By examining these differences, it becomes possible to assess alterations in autonomic function and identify how stroke can disrupt normal cardiovascular control.

The boxplot 1319 of FIG. 13J illustrates the distribution of Autoregressive (AR) relative power VLF values in stroke patients and healthy individuals. AR relative power VLF is a frequency-domain measure of heart rate variability that focuses on the very low-frequency (VLF) components, which reflect long-term regulatory processes such as thermoregulation and hormonal influences. The VLF power can be associated with autonomic balance, with higher values indicating stronger autonomic activity, while lower values suggest impaired regulation. The boxplot 1319 can compare the distribution of AR relative power VLF values across stroke patients and healthy individuals, showing potential differences in autonomic function. Analyzing these variations can help assess disruptions in cardiovascular control linked to stroke, such as altered sympathetic and parasympathetic responses.

As shown in FIG. 13K, the boxplot 1321 illustrates the distribution of M values in stroke patients and healthy individuals. The M value is a nonlinear measure of heart rate variability that reflects the complexity and irregularity of the heart rate signal. It can be used to assess the adaptability of the autonomic nervous system, with higher values indicating greater complexity in heart rate dynamics, while lower values suggest reduced autonomic regulation. The boxplot 1321 can compare the distribution of M values across stroke patients and healthy individuals, showing potential differences in autonomic function. Analyzing these variations can help assess disruptions in cardiovascular control linked to stroke, such as altered sympathetic and parasympathetic responses.

In FIG. 13L, the boxplot 1323 illustrates the distribution of DFA α2 values in stroke patients and healthy individuals. DFA α2 is a parameter obtained through detrended fluctuation analysis (DFA), which evaluates the scaling behavior of heart rate variability over different time scales. This DFA α2 reflects the self-similarity of heart rate fluctuations, with higher DFA α2 values indicating a more adaptable and balanced autonomic regulation, while lower values suggest a loss of complexity that can be associated with autonomic dysfunction. The boxplot 1323 contrasts DFA α2 values between stroke patients and healthy individuals, showing potential differences in the regulation of heart rate dynamics. Variations in DFA α2 can provide insight into how stroke affects cardiovascular control, including disruptions in sympathetic and parasympathetic balance.

The boxplot 1325 of FIG. 13M illustrates the distribution of Poincaré SD2 values in stroke patients and healthy individuals. Poincaré SD2 is a time-domain measure of heart rate variability that quantifies the long-term variability of consecutive RR intervals. It reflects the overall variability in heart rate and is often associated with the parasympathetic nervous system's influence on heart rate regulation. Higher SD2 values indicate greater variability and more flexible autonomic control, while lower values suggest reduced autonomic responsiveness and potential dysfunction. The boxplot 1325 contrasts Poincaré SD2 values between stroke patients and healthy individuals, revealing potential differences in autonomic regulation. Variations in SD2 values can indicate disruptions in the autonomic nervous system, providing insight into the cardiovascular disturbances commonly observed in stroke patients.

As shown in FIG. 13N, the boxplot 1327 illustrates the distribution of Welch total power values in stroke patients and healthy individuals. Welch total power is a frequency-domain measure of heart rate variability that quantifies the overall power across all frequency bands, providing a comprehensive view of autonomic nervous system activity. Higher total power values indicate greater overall heart rate variability, which reflects more balanced autonomic regulation, while lower values suggest reduced variability and possible autonomic dysfunction. The boxplot 1327 compares Welch total power values between stroke patients and healthy individuals, highlighting potential differences in autonomic control. Variations in total power can reveal disruptions in the sympathetic and parasympathetic balance, offering insight into cardiovascular disturbances commonly associated with stroke.

### Terminology

In the foregoing specification, the systems and processes have been described with reference to specific implementations thereof. It will, however, be evident that various modifications and changes may be made thereto without departing from the broader spirit and scope of the implementations disclosed herein. The specification and drawings are, accordingly, to be regarded in an illustrative rather than restrictive sense.

Indeed, although the systems and processes have been disclosed in the context of certain implementations and examples, it will be understood by those skilled in the art that the various implementations of the systems and processes extend beyond the specifically disclosed implementations to other alternative implementations and/or uses of the systems and processes and obvious modifications and equivalents thereof. In addition, while several variations of the implementations of the systems and processes have been shown and described in detail, other modifications, which are within the scope of this disclosure, will be readily apparent to those of skill in the art based upon this disclosure. It is also contemplated that various combinations or sub-combinations of the specific features and implementations of the implementations may be made and still fall within the scope of the disclosure. It should be understood that various features and implementations of the disclosed implementations can be combined with, or substituted for, one another in order to form varying modes of the implementations of the disclosed systems and processes. Any methods disclosed herein need not be performed in the order recited. Thus, it is intended that the scope of the systems and processes herein disclosed should not be limited by the particular implementations described above.

It will be appreciated that the systems and methods of the disclosure each have several innovative implementations, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate implementations also may be implemented in combination in a single implementations. Conversely, various features that are described in the context of a single implementation also may be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every implementation.

Unless the context clearly requires otherwise, throughout the description and the claims, the words “comprise,” “comprising,” “include,” “including” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Moreover, as used herein, when a first element is described as being “on” or “over” a second element, the first element may be directly on or over the second element, such that the first and second elements directly contact, or the first element may be indirectly on or over the second element such that one or more elements intervene between the first and second elements. Where the context permits, words in the above Detailed Description using the singular or plural number may also include the plural or singular number, respectively. The word “or” in reference to a list of two or more items, that word covers all of the following interpretations of the word: any of the items in the list, all of the items in the list, and any combination of the items in the list.

Moreover, conditional language used herein, such as, among others, “can,” “could,” “might,” “may,” “e.g.,” “for example,” “such as” and the like, unless specifically stated otherwise, or otherwise understood within the context as used, is generally intended to convey that certain implementations include, while other implementations do not include, certain features, elements and/or states. Thus, such conditional language is not generally intended to imply that features, elements and/or states are in any way required for one or more implementations.

While certain implementations have been described, these implementations have been presented by way of example only, and are not intended to limit the scope of the disclosure. Indeed, the novel apparatus, methods, and systems described herein may be embodied in a variety of other forms; furthermore, various omissions, substitutions and changes in the form of the methods and systems described herein may be made without departing from the spirit of the disclosure. For example, while blocks are presented in a given arrangement, alternative implementations may perform similar functionalities with different components and/or circuit topologies, and some blocks may be deleted, moved, added, subdivided, combined, and/or modified. Each of these blocks may be implemented in a variety of different ways. Any suitable combination of the elements and acts of the various implementations described above can be combined to provide further implementations. The accompanying claims and their equivalents are intended to cover such forms or modifications as would fall within the scope and spirit of the disclosure.

Several illustrative examples of systems and methods for detecting an anomalous biologic event in a person have been disclosed. Although this disclosure has been described in terms of certain illustrative examples and uses, other examples and other uses, including examples and uses which do not provide all of the features and advantages set forth herein, are also within the scope of this disclosure. Components, elements, features, acts, or steps may be arranged or performed differently than described and components, elements, features, acts, or steps may be combined, merged, added, or left out in various examples. All possible combinations and subcombinations of elements and components described herein are intended to be included in this disclosure. No single feature or group of features is necessary or indispensable.

Certain features that are described in this disclosure in the context of separate implementations may also be implemented in combination in a single implementation. Conversely, various features that are described in the context of a single implementation also may be implemented in multiple implementations separately or in any suitable subcombination. Moreover, although features may be described above as acting in certain combinations, one or more features from a claimed combination may in some cases be excised from the combination, and the combination may be claimed as a subcombination or variation of a subcombination.

Further, while illustrative examples have been described, any examples having equivalent elements, modifications, omissions, and/or combinations are also within the scope of this disclosure. Moreover, although certain aspects, advantages, and novel features are described herein, not necessarily all such advantages may be achieved in accordance with any particular example. For example, some examples within the scope of this disclosure achieve one advantage, or a group of advantages, as taught herein without necessarily achieving other advantages taught or suggested herein. Further, some examples may achieve different advantages than those taught or suggested herein.

Some examples have been described in connection with the accompanying drawings. The figures may or may not be drawn and/or shown to scale, but such scale should not be limiting, since dimensions and proportions other than what are shown are contemplated and are within the scope of the disclosed devices, systems, and methods. Distances, angles, etc. are merely illustrative and do not necessarily bear an exact relationship to actual dimensions and layout of the devices illustrated. Components may be added, removed, and/or rearranged. Further, the disclosure herein of any particular feature, aspect, method, property, characteristic, quality, attribute, element, or the like in connection with various examples may be used in all other examples set forth herein. Additionally, any methods described herein may be practiced using any device suitable for performing the recited steps.

For purposes of summarizing the disclosure, certain aspects, advantages and features of several devices, systems, and methods have been described herein. Not all, or any such advantages are necessarily achieved in accordance with any particular example of the devices, systems, and methods disclosed herein. No aspects of this disclosure are essential or indispensable. In many examples, the devices, systems, and methods may be configured differently than illustrated in the figures. or description herein. For example, various functionalities provided by the illustrated modules may be combined, rearranged, added, or deleted. In some implementations, additional or different processors or modules may perform some or all of the functionalities described with reference to the examples described and illustrated in the figures. Many implementation variations are possible. Any of the features, structures, steps, or processes disclosed in this specification may be included in any example.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain implementations require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the implementations shown herein but are to be accorded a fair interpretation consistent with this disclosure, the principles and the novel features disclosed herein.

Depending on the implementation, certain acts, events, or functions of any of the methods described herein can be performed in a different sequence, can be added, merged, or left out altogether (e.g., not all described acts or events are necessary for the practice of the method). Moreover, in certain implementations, acts or events can be performed concurrently, e.g., through multi-threaded processing, interrupt processing, or multiple processors or processor cores, rather than sequentially.

The various illustrative logical blocks, modules, circuits, and algorithm steps described in connection with the implementations disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks, modules, and circuits described in connection with the implementations disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the implementations disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features as applied to various implementations, it will be understood that various omissions, substitutions, and changes in the form and details of the devices or algorithms illustrated can be made without departing from the spirit of the disclosure. As will be recognized, certain implementations of the devices, systems, and methods described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A system for detecting an anomalous biologic event in a person, the system comprising:
a wearable device comprising at least one physiological sensor configured to monitor at least one tissue site of the person and a stimulus source configured to apply a stimulus at the at least one tissue site to invoke a physiological response at the least one tissue site; and
at least one hardware processor configured to:
receive first data associated with the person;
cause the stimulus source to generate a stimulus at the at least one tissue site;
cause operation of the at least one physiological sensor to trigger monitoring of the at least one tissue site to generate second data, wherein the first data comprises at least one type of physiological parameter data that is different from the second data;
extract a set of features from the first data and the second data;
generate a classifier based on at least two of the extracted set of features, wherein at least one of the extracted set of features comprises a metric corresponding to heart rate variability; and
determine a likelihood of detection of the anomalous biologic event based at least in part on the generated classifier.

2. The system of claim 1, wherein the stimulus source comprises a heater and the second data comprises skin temperature.

3. The system of claim 2, wherein the first data comprises a data associated with heart activity of the person.

4. The system of claim 1, wherein the second data comprises data associated with a skin temperature at two different locations on a body of the person.

5. A system for detecting an anomalous biologic event in a person, the system comprising:
a wearable device comprising at least one physiological sensor configured to monitor at least one tissue site of the person and a stimulus source configured to apply a stimulus at the at least one tissue site to invoke a physiological response at the least one tissue site; and
at least one hardware processor configured to:
cause operation of the at least one physiological sensor to trigger monitoring of the at least one tissue site to generate first data;
extract at least one feature from the first data;
generate a classifier based on the extracted feature from the first data;
compare the at least one feature to the generated classifier and a first threshold, wherein the first threshold is based at least in part on patient specific information;
in response to a determination that the at least one feature satisfies the generated classifier and passes the first threshold, output an alert of a detection of the anomalous biologic event; and
in response to a determination that the at least one feature does not satisfy the generated classifier and pass the first threshold, cause operation of the at least one physiological sensor to continue monitoring of the at least one tissue site to generate second data.

6. The system of claim 5, wherein the stimulus source comprises a heater and the second data comprises skin temperature.

7. The system of claim 6, wherein the first data comprises a data associated with heart activity of the person.

8. The system of claim 5, wherein the second data comprises data associated with a skin temperature at two different locations on a body of the person.

9. A system for detecting an anomalous biologic event in a person, the system comprising:
a wearable device comprising at least one physiological sensor configured to monitor at least one tissue site of the person and a stimulus source configured to apply a stimulus at the at least one tissue site to invoke a physiological response at the least one tissue site; and
at least one hardware processor configured to:
receive first data associated with the person;
cause the stimulus source to generate a stimulus at the at least one tissue site;
cause operation of the at least one physiological sensor to trigger monitoring of the at least one tissue site to generate second data, wherein the first data comprises at least one type of physiological parameter data that is different from the second data;
extract a first and second set of features from the first data and the second data;
generate a classifier based on at least the first and second set of features from the first data and the second data;
determine a first likelihood of detection of the anomalous biologic event based on the first set of features and the generated classifier;
determine a second likelihood of detection of the anomalous biologic event based on the second set of features and the generated classifier;
determine an overall likelihood of detection of the anomalous biologic event based at least in part on the first likelihood and the second likelihood; and
output an alert in response to the overall likelihood.

10. The system of claim 9, wherein the at least one hardware processor is configured to cause the at least one physiological sensor to measure a distal temperature at a sampling rate of 16 Hz.

11. The system of claim 9, further comprising generating an alert based at least in part on the likelihood of detection of the anomalous biologic event.

12. The system of claim 9, wherein the tissue site comprises a wrist.

13. The system of claim 9, wherein the stimulus source comprises a heater and the second data comprises skin temperature.

14. The system of claim 13, wherein the first data comprises a data associated with heart activity of the person.

15. The system of claim 9, wherein the second data comprises data associated with a skin temperature at two different locations on a body of the person.

16. A system for detecting an anomalous biologic event in a person, the system comprising:
a wearable device comprising at least one physiological sensor configured to monitor at least one tissue site of the person;
a memory; and
at least one hardware processor configured to:
cause to continuously measure, during a sleep period of the person by the at least one physiological sensor, a distal temperature at the at least one tissue site to generate a plurality of temperature data;
extract a set of features from the plurality of temperature data;
generate a classifier based on at least the set of features from the plurality of temperature data; and
determine a likelihood of detection of the anomalous biologic event based at least in part on a the generated classifier.

17. The system of claim 16, wherein an anomalous medical event is associated with increased variation.

18. The system of claim 16, wherein the at least one hardware processor is configured to cause the at least one physiological sensor to measure a distal temperature at a sampling rate of 16 Hz.

19. The system of claim 16, wherein the comparison comprises:
determining a presence or absence of an average increase in temperature values during the sleep period in the plurality of temperature data;
determining a variation in temperature values during the sleep period in the plurality of temperature data; and
generating the likelihood of detection of an anomalous medical event based at least in part on the absence of the average increase in temperature values and the variation in temperature values.

20. The system of claim 19, wherein an anomalous medical event is associated with an absence the average increase in temperature values during the sleep period.
